(19) United States
(12) Patent Application Publication
(10) Pub. No.: US 2010/0184963 A1
(43) Pub. Date: Jul. 22, 2010

(54) QUINOA PROTEIN CONCENTRATE, PRODUCTION AND FUNCTIONALITY

(75) Inventors: Laurie A. Scanlin, Arvada, CO (US); Martha B. Stone, Fort Collins, CO (US); Claire Burnett, Erie, CO (US)

Correspondence Address: SMITH HOPEN, PA, 180 PINE AVENUE NORTH, OLDSMAR, FL, 34677, US

(73) Assignee: KEEN INGREDIENTS, INC,, Louisville, CO (US)

(21) Appl. No.: 12/748,968

(22) Filed: Mar. 29, 2010

Related U.S. Application Data
(63) Continuation of application No. PCT/US2008/078111, filed on Sep. 29, 2008.
(60) Provisional application No. 60/975,653, filed on Sep. 27, 2007.
(60) Provisional application No. 60/975,637, filed on Sep. 27, 2007.
(60) Provisional application No. 60/978,293, filed on Oct. 8, 2007.
(60) Provisional application No. 60/978,311, filed on Oct. 8, 2007.
(60) Provisional application No. 60/978,328, filed on Oct. 8, 2007.
(60) Provisional application No. 60/978,339, filed on Oct. 8, 2007.
(60) Provisional application No. 60/978,348, filed on Oct. 8, 2007.
(60) Provisional application No. 60/978,352, filed on Oct. 8, 2007.

(51) Int. Cl. C07K 1/14; C07K 1/30; C07K 1/34; C07K 1/36; C08B 31/00; C11B 1/10

(52) U.S. Cl. 530/412; 536/102; 554/8

(57) ABSTRACT

The present invention relates to a new source of high quality plant protein, termed, “quinoa protein concentrate” or “QPC”, which contains at least about 50 wt % protein which is food-grade and/or pharmaceutical-grade and methods of preparing such protein concentrates as well as starch, oil, and fiber from quinoa grain. The quinoa protein concentrate of the invention is useful as food ingredients, infant formula ingredients, cosmetic ingredients, pet food ingredients, and animal feed supplements.

### CROSS REFERENCE TO RELATED APPLICATIONS

This application is a continuation of International Application No. PCT/US2008/078111, filed Sep. 29, 2008, which claims priority to (1) U.S. Provisional Patent Application 60/975,653, entitled, “Methods for Manufacturing Edible Quinoa Flour Having Improved Flavor, Shelf-Life, Rheological, Cooking, and Baking Characteristics and Uses Thereof”, filed Sep. 27, 2007, (2) U.S. Provisional Patent Application 60/975,637, entitled, “Methods for Manufacturing Edible Quinoa Germ Having Concentrated Nutrient Profile, Novel Texture, Acceptable Flavor, Cooking, and Baking Characteristics and Uses Thereof”, filed Sep. 27, 2007, (3) U.S. Provisional Patent Application 60/978,293, entitled, “Methods for Manufacturing Edible Quinoa Oil and Uses Thereof”, filed Oct. 8, 2007, (4) U.S. Provisional Patent Application 60/978,311, entitled, “Methods for Manufacturing Edible Quinoa Protein Concentrate or Quinoa Protein Isolate and Uses Thereof”, filed Oct. 8, 2007, (5) U.S. Provisional Patent Application 60/978,328, entitled, “Methods for Manufacturing Unmodified Quinoa Starch and Uses Thereof”, filed Oct. 8, 2007, (6) U.S. Provisional Patent Application 60/978,339, entitled, “Method for Treating Quinoa Grain and the Edible Product Obtained Having Novel Texture, Flavor, and Extended Shelf-Life and Uses Thereof”, filed Oct. 8, 2007, (7) U.S. Provisional Patent Application 60/978,348, entitled, “Method for Treating Quinoa Grain and the Edible Sweet Liquid and Sweet Powdered Product Obtained and Uses Thereof”, filed Oct. 8, 2007, (8) and to U.S. Provisional Patent Application 60/978,352, entitled, “Methods for Manufacturing Edible Quinoa Fiber and Uses Thereof”, filed Oct. 8, 2007, the contents of which are herein incorporated by reference. International Application No. PCT/US2008/078111 is further a continuation-in-part of application Ser. No. 10/596,259, filed Sep. 20, 2006, which is the National Stage of International Application No. PCT/US2004/042296, filed Dec. 16, 2004, which claims priority to U.S. Provisional Patent Application 60/530,219, entitled, “Quinoa Protein Concentrate, Production and Functionality”, filed Dec. 16, 2003, the contents of which applications are herein incorporated by reference.

### FIELD OF INVENTION

The present invention relates to a quinoa protein concentrate and a methods of processing quinoa (Genus: Chenopodium, Species: quinoa, Family: Chenopodiaceae) grain (also called quinoa seed, quinua, grain-like seed, pseudocereal, and fruit) to produce such protein concentrate, flour, germ, oil, starch, and fiber.

### BACKGROUND OF THE INVENTION

Plant proteins, processed from cereal grains and legumes, are profitable ingredients in a wide variety of commercial food products, pet foods, and animal feed. Examples of the plant proteins that are currently available are soy protein concentrate, isolated soy protein, wheat gluten, rice, and corn proteins [Food Master (2003) Ingredients and R&D services catalog. Bensenville Ill. Business News Publishing Co. II. LLC].

However, plant proteins are often limiting in one or more essential amino acids. For example, the plant proteins of wheat, rice and corn are limiting in lysine [Hoseney, R C (1986) In: Principles of cereal science and technology. St. Paul, Minn.: American Association of Cereal Chemists, Inc. ppg. 69-88], whereas, soy protein is limiting in methionine and cystine [Haard and Chism (1996) In: Fennema O R, editor. Food Chemistry, 3^{rd} ed. revised and expanded. New York: Marcel Dekker, ppg. 943-1011]. Well-processed isolated soy proteins and soy protein concentrates have been found to be equivalent to animal protein in regard to the needs of human nutrition [Young, V R (1991) J. Am. Diet Assoc. 7: ppg. 828-835].

The following eight foods, while they are a good source of animal or plant protein, account for 90% of all food allergenic reactions: soy, wheat, eggs, milk, peanuts, tree nuts, fish and shellfish [Hefle, S. L. et al. (1996) Crit. Rev. Food Sci. Nutr. 36(5): ppg. 69-89]. Food allergens are a serious concern leading individuals susceptible to food allergenic reactions to adopt a restricted diet that can be lacking in essential nutrients for proper health due to a narrowed food choice. In addition, there remains the life-threatening concern of anaphylactic shock in highly sensitive individuals due to exposure, which is frequently unintended, to food allergens. Allergens are problematic for food producers because many food ingredients fall into this category and limit product development. The impact that food allergens, including undeclared food allergens, have had on the food industry is significant and the FDA has made food allergens are a top priority in recent years [Hefle, S. (September 2003) Symposium: Update on Food Allergens. American Association of Cereal Chemists Annual Meeting. Portland, Oreg.].

As world food demands steadily increase, production of protein has to be maximized, as well as augmented. Plant proteins from cereals and legumes represent the main source of proteins and energy supply for both human and animal nutrition. This is partly due to the fact that animal proteins require much higher energy demand for production and are therefore more expensive to produce than plant proteins [Cheftel, J C et al. (1985) In: Fennema Oreg., editor. Food Chemistry, 2^{nd} ed. New York: Marcel Dekker. ppg. 245-369]. For example, in order to produce 1 kg of animal protein, 3-20 kg of plant protein is needed. Consequently, as demands for animal protein increase globally, the need for plant protein increases drastically. To meet this need, new protein resources must be developed. Protein-rich crops that give equitable yields in underutilized growing regions are of paramount value for this purpose. Alternatively, new crops can be selected and tested for a protein source.

Since 1975, quinoa has become an alternative crop in North America and Europe for many reasons including that (1) quinoa has the ability to thrive in marginal soils, where traditional crops cannot, therefore, underutilized growing regions can be cultivated; (2) quinoa has an average protein content of 14.6%, which is higher than traditional cereals, with certain varieties containing protein levels as high as 21.9%; (3) and quinoa has an amino acid composition, protein efficiency ratio, protein digestibility, and nitrogen balance comparable to milk protein, casein [Fleming and Galwey (1995) In: Williams, J T, editor. Underutilized Crops: Cereals and Pseudocereals. New York: Chapman and Hall, ppg. 3-83]. Few plant proteins so closely resemble that of animal origin as quinoa protein.

Quinoa protein is particularly high in lysine and methionine, amino acids limiting in cereal grains and legumes, respectively [Koziol, M J (1992) J. Food Composition and Analysis 5: ppg. 35-68]. Quinoa protein is also high in histidine, an essential amino acid for infant development and those with chronic diseases [Ettinger, S (2000) In: Mahan K L, Escott-Stump S, eds. Krause's Food, Nutrition, and Diet Therapy, 10^{th} ed. Philadelphia, Pa. WB Saunders Co. ppg. 54-61]. In South America, it has been used as a weaning food for centuries because of its nutritional attributes and high protein digestibility.

Additionally, quinoa is not on the list of recognized food allergens. It is considered free of gluten or prolamins [Fairbanks, O J et al. (1990) Plant Breeding 104(3): ppg. 190-195], the proteins associated with allergenic reactions in wheat gluten, rye and barley. Prolamins, like gliadins found in wheat, trigger immune responses in patients with gluten-induced enteropathy, also known as celiac disease. Quinoa is a pseudocereal named for its production of small grain-like seeds, although the actual harvested grain is a single seeded fruit [Shewry, P R (2002) In: Belton P S, Taylor J. eds. Pseudocereals and Less Common Cereals. Germany: Springer-Verlag Berlin Heidelberg. ppg. 93-122]. It is a dicotyledonous species not closely related to the monocotyledonous species of true cereal grains like wheat, rye, and barley. As a result of differences in plant taxonomy, quinoa does not contain the harmful amino acid sequences found in wheat. Therefore, it is concluded safe for a gluten-free diet [Thompson, T. (2001) J. Am. Diet. Assoc. 101: ppg. 586-587] and is recommended by the Celiac Disease Foundation and Gluten Intolerance Group. Furthermore, research presented at the International Workshop on Food Supplementation in Food Allergy and Immunity, found that quinoa is immunochemically safe and represents a viable alternative for gluten-free products [Berti, C et al. (August 2002) International Workshop on Food Supplementation in Food Allergy and Immunity. Olsztyn].

Despite the numerous beneficial properties of quinoa as a plant protein source as described above, quinoa grain has not been processed efficiently to extract individual components contained therein. Currently, quinoa is available only as whole grain or ground for a small number of products. Therefore, there is a need in the art to develop a method to process quinoa grains into individual components, i.e., protein, oil, fiber, and starch, which are food-grade and/or pharmaceutical-grade that can readily be utilized as nutritional supplements as well as agents for providing functionality in a variety of food products, cosmetic products, and animal feeds. The present invention meets this important need. The advantages of the invention will be evident in the following description.

### SUMMARY OF INVENTION

The present invention provides a new source of plant protein, termed “quinoa protein concentrate” (QPC), prepared from quinoa (Chenopodium quinoa Chenopodiaceae) grain, which, in an advantageous embodiment, contains at least about 50 wt % protein, preferably at least about 70 wt % protein, most preferably at least about 90 wt % protein, on a dry weight basis.

The QPC of the invention is high in lysine and histidine, which are often limiting in plant proteins of grains. The QPC of the invention is also high in methionine and cystine, which are often limiting in legumes. Additionally, quinoa is considered to be hypo-allergenic (even non-allergenic), as opposed to key plant allergens, soy and wheat. Therefore, the quinoa protein concentrate is useful as food ingredients and supplements to provide nutrients as well as necessary functionality in a variety of food products including infant formula, pet foods and animal feeds. For example, the QPC can be added in a variety of products such as foods for infants and toddlers, meat analogs, ice creams, whipped toppings, baked products, and salad dressings and the like, to reduce water activity, reduce fat, bind ingredients, emulsify, and/or stabilize foams. The QPC of the invention are particularly useful as an ingredient to fortify the amino acid composition of corn- or rice-based food products, which are also considered to be hypo-allergenic, but are either low in protein content or limiting in essential amino acid, lysine. The QPC can be used as a protein source in food or cosmetic products intended for use in subjects who require less- or hypo-allergenic food products.

In addition, QPC can serve as a high quality, plant protein in pet foods and animal feeds like cattle feed. The opportunity afforded by this use is has become since the FDA banned the use of animal protein in cattle feed due to concerns over bovine spongiform encephalopathy (i.e., BSE or mad cow disease) [DEPARTMENT OF HEALTH AND HUMAN SERVICES (2004), Food and Drug Administration, 21 CFR Parts 189 and 700, [Docket No. 2004N-0081], RIN-0910-AF47, Use of Materials Derived From Cattle in Human Food and Cosmetics].

Also provided are processes for isolating individual components contained in quinoa (Chenopodium quinoa Chenopodiaceae) grain such as protein (termed QPC herein), oil, starch, and fiber. In an advantageous embodiment a process comprises the steps of; 1) flaking or comminuting quinoa grain, 2) extracting oil from the flaked or comminuted quinoa grain leaving defatted quinoa, 3) extracting protein from the defatted quinoa in alkaline solution, 4) separating the fraction containing the protein from the mixture, and 5) drying the solubilized protein, whereby a quinoa protein concentrate containing at least about 50 wt % protein is obtained. The term, “comminution” or “comminuting”, is generically used herein to indicate a step of treatment such as grinding, milling, disintegration, trituration, pulverization, etc. Quinoa oil, fiber, and starch can be readily obtained from this process by employing simple manipulations such as separation or concentration.

In a second aspect of the processes for isolating individual components contained in quinoa, the present invention provides a method of processing quinoa grain employing the steps of: pre-conditioning the quinoa grain; conditioning the pre-conditioned quinoa grain; comminuting the conditioned quinoa grain; and separating the comminuted quinoa grain. The separation step yields a germ-rich fraction and a peri-sperm rich fraction.

The pre-conditioning step can utilize mechanical abrasion, washing the quinoa grain and combinations thereof. In an advantageous embodiment the pre-conditioning includes abrasion followed by an initial quick wash with stirring, agitation, spray or counter current extraction followed by draining or centrifugation. The quick wash both (1) removes saponins and (2) minimizes penetration of the water-soluble saponins into the grain. In a particularly advantageous embodiment the pre-conditioning includes abrasion followed by a plurality of quick washes. One or more of the plurality of quick washes can include stirring, agitation, spray or counter current extraction followed by draining or centrifugation, again minimizing penetration of water-soluble saponins into grain. The first of the plurality of quick washes can employ a residence time of about 30 seconds to about 2 minutes and a subsequent wash can employ a residence time of about 2 minutes to about 10 minutes. Such a washing schema is found to be particularly effective in reducing the presence of saponins in the grain. Alternatively, the pre-conditioning can utilize techniques such as mechanical abrasion, washing, polishing, peeling, aspiration, air classification, sieving, pneumatic pressure, vacuum, nixtamalization, rinsing, solvent leaching the quinoa grain and combinations thereof.

The conditioning step can be performed by evaporating water from the grain to yield grain with a moisture content of about 12 to about 30%. In an advantageous embodiment the grain is conditioned to a moisture content of about 13 to about 14%.

In still further advantageous embodiments the method can include the step of separating the comminuted quinoa grain prior to protein extraction. The separation step yields a germ-rich fraction and a perisperm-rich fraction. Quinoa protein can then be isolated from the germ-rich fraction and the perisperm-rich fraction can be put to other uses, such as the production of quinoa flour and/or quinoa starch. Techniques suitable for isolating the quinoa protein include extraction, purification, iso-electric precipitation, ultra-filtration and concentration.

The process of the second aspect can include the step of isolating the quinoa protein from the germ-rich fraction. Techniques for isolating the quinoa protein from the germ-rich fraction can include of extraction, purification, iso-electric precipitation, ultra-filtration and concentration. The extracted protein can be clarified to remove quinoa fiber or impurities. In an advantageous embodiment the extracted protein is clarified by centrifugation. The extracted protein can be neutralized (i.e. the pH adjusted) to neutralize the alkaline solution used for the extraction. Further purification and solidification of the quinoa protein can be achieved by precipitating the neutralized protein, isolating the precipitated protein and neutralizing the precipitated protein. The neutralized, precipitated protein can then be dried where a dried, finished product is desired.

As discussed above, the process of the second aspect employs the step of comminuting the conditioned quinoa grain. Quinoa grain can be comminuted by polishing, abrasion, milling, pin milling, hammer milling, degerming, stone grinding, cracking, crushing, slicing, flaking and combinations thereof. The comminuted quinoa grain can then be separated using a technique such as sieving, aspiration, air classification, pneumatic pressure, vacuum, vibration and combinations thereof.

The process of the second aspect can further include the step of isolating the quinoa oil from the germ-rich fraction. Quinoa oil can be isolated from the germ-rich fraction using techniques such as purification, mechanical pressing, supercritical gas-extraction, solvent-extraction and combinations thereof. The isolated quinoa oil can further be treated to produce the finished product by refining, deodorizing, bleaching, enzyme modification, chemical hydrolysis and combinations thereof.

Additionally, the process can include the step of isolating the quinoa fiber from the germ-rich fraction. Techniques for isolating the quinoa fiber include purification, sieving, filtering, flocculation, centrifuging, mechanical pressing, supercritical gas-extraction, or liquid-extraction, air-classification, aspiration and combinations thereof. The isolated quinoa fiber can further be treated to produce the finished product by refining, deodorizing, bleaching, enzyme modification, chemical hydrolysis and combinations thereof.

In certain embodiments the process of the second aspect can also include the step of isolating the quinoa starch from the perisperm-rich fraction. Techniques for isolating quinoa starch from the perisperm-rich fraction include purification, extraction, and combinations thereof. The isolated quinoa fiber can further be treated to produce the finished product by refining, deodorizing, bleaching, enzyme modification, chemical hydrolysis and combinations thereof.

In a third aspect the present invention provides a method of processing quinoa grain having the steps of comminuting the quinoa grain and extracting the protein from the comminuted quinoa grain using an alkaline solution. The alkaline solution solubilizes the protein from the comminuted quinoa grain.

In an advantageous embodiment the method further includes the step of clarifying the extracted protein to remove quinoa fiber or impurities. The extracted protein can be clarified by centrifugation.

In further advantageous embodiments, the method includes the step of neutralizing the extracted protein. Thus, a quinoa protein is produced in a liquid format. The neutralized protein then be precipitated, isolated (such as by centrifugation) and further neutralized.

The method can also include the step of extracting quinoa oil from the quinoa grain. The oil can be extracted following the comminuting step.

In an advantageous embodiment the method further includes the steps of pre-conditioning the quinoa grain and conditioning the pre-conditioned quinoa grain prior to the comminuting step. As above, one benefit of the pre-conditioning is the removal of saponins from the grain.

In still further advantageous embodiments the method can include the step of separating the comminuted quinoa grain prior to protein extraction. The separation step yields a germ-rich fraction and a perisperm-rich fraction. Quinoa protein can then be isolated from the germ-rich fraction and the perisperm-rich fraction can be put to other uses, such as the production of quinoa flour and/or quinoa starch. Techniques suitable for isolating the quinoa protein include extraction, purification, iso-electric precipitation, ultra-filtration and concentration.

Techniques for pre-conditioning quinoa grain include mechanical abrasion, washing the quinoa grain and combinations thereof. An advantageous pre-conditioning includes abrasion followed by a plurality of quick washes.

Techniques for conditioning quinoa grain include evaporating water from the grain to yield grain with a moisture content of about 12 to about 30%. Advantageously, the grain is conditioned to a moisture content of about 13 to about 14%.

Additionally, quinoa oil can be isolated from the germ-rich fraction produced in the separation step. Quinoa fiber can also be isolated from the germ-rich fraction. In a similar fashion, quinoa starch can be isolated from the perisperm-rich fraction.

In a fourth aspect the present invention provides a method of processing quinoa grain using the steps of pre-conditioning the quinoa grain, conditioning the pre-conditioned quinoa grain, comminuting the conditioned quinoa grain, separating the comminuted quinoa grain and isolating the quinoa protein from the germ-rich fraction. Techniques for isolating the quinoa protein include extraction, purification, iso-electric precipitation, ultra-filtration and concentration. The extracted protein can then be clarified to remove quinoa fiber or impurities. Clarification can be performed by centrifugation. The extracted protein can be neutralized (i.e. the pH adjusted) to neutralize the alkaline solution used for the extraction. Further purification and solidification of the quinoa protein can be achieved by precipitating the neutralized protein, isolating the precipitated protein and neutralizing the precipitated protein. Additionally, the collected perisperm-rich fraction can be further processed for use as quinoa flour or quinoa starch.

It will be understood by those skilled in the art that the processes disclosed herein can be operated with appropriate modifications and variations to obtain the afore-mentioned products. For example, with respect to the first aspect, the quinoa grain can be mechanically abraded prior to the step of comminution and/or the quinoa grain can be shaped (such as flaked) prior to the step of comminution, and/or the quinoa grain can be conditioned (such as tempered) prior to the step of comminution. The protein fraction obtained after step (4) can be further purified by isoelectric precipitation before step (5), if necessary. The process disclosed herein is designed to maximize isolation of the individual components contained in quinoa grain and thus enables one to obtain other components such as quinoa oil, starch, and fiber at different stages of the process, as illustrated in the flow diagrams of the figures below.

### BRIEF DESCRIPTION OF THE DRAWINGS

For a fuller understanding of the invention, reference should be made to the following detailed description, taken in connection with the accompanying drawings, in which:

FIG. 1 is a flowchart (Scheme 1) illustrating the preparation quinoa protein concentrate from Chenopodium quinoa, Chenopodiaceae.

FIG. 2 is a flowchart (Scheme 2) illustrating an alternative method to the exemplary method of FIG. 1 for the preparation quinoa protein concentrate from Chenopodium quinoa, Chenopodiaceae.

FIG. 3 is a flowchart (Scheme 3) illustrating an alternative method to the exemplary method of FIG. 1 for the preparation quinoa protein concentrate from Chenopodium quinoa, Chenopodiaceae.

FIG. 4 is a flowchart illustrating an exemplary method of making edible quinoa germ and edible quinoa flour from Chenopodium quinoa, Chenopodiaceae.

FIG. 5 is a flowchart illustrating an exemplary method of making edible quinoa oil from Chenopodium quinoa, Chenopodiaceae.

FIG. 6 is a flowchart illustrating an alternative method to the exemplary method of FIG. 1 for the preparation quinoa protein concentrate from Chenopodium quinoa, Chenopodiaceae.

FIG. 7 is a flowchart illustrating an exemplary method of making edible quinoa fiber from Chenopodium quinoa, Chenopodiaceae.

FIG. 8 is a flowchart illustrating an exemplary method of making edible quinoa starch from Chenopodium quinoa, Chenopodiaceae.

### DETAILED DESCRIPTION OF THE PREFERRED EMBODIMENT

In general the terms and phrases used herein have their art-recognized meaning, which can be found by reference to standard texts, journal references and contexts known to those skilled in the art. The following definitions are provided to clarify their specific use in the context of the invention.

The term, “quinoa protein concentrate (QPC)”. as used herein, is intended to indicate the product obtained from quinoa (Genus: Chenopodium, Species: quinoa, Family: Chenopodiaceae) grain (also called quinoa seed, grain-like seed, pseudocereal, and fruit) having a protein content of at least about 50 wt %, preferably of at least about 70 wt %, most preferably of at least about 90 wt %, on a dry weight basis, and is food- and pharmaceutical-grade. The QPC can be obtained by the processes disclosed herein with or without modifications. The protein content is determined by the procedure as described in American Association of Cereal Chemists:” Approved Methods of Analysis,” The Association, St. Paul, Minn., 2000. However, any art-recognized methods can be used to determine the protein content in the product obtained by the process of the invention. Typically, the percentage of the protein content on a dry weight basis is determined by kjeldahl nitrogen×6.25 (N×6.25).

The term, “functionality”, is a well-known term in the food industry and relates to physical and chemical properties of food molecules that affect their behavior and produce desired effects in foods during formulation, processing, preparation, and storage [Murano, P S (2003) Understanding Food Science and Technology. Belmont, Calif.: Wadsworth/Thomson Learning, Inc.]

The term, “infant food”, more commonly referred to as “food for infants”, means any food product intended for use for infants up to one year in age, and generally refers to solid foods for older infants age six months to one year in age. “Foods for toddlers” generally refers to foods for toddlers age one year to two years in age. “Foods for children” refers to foods for pre-school children age 2-5 years and schoolchildren up to 12 years in age. The designation becomes important when estimating amino acid requirements.

When a Markush group or other grouping is used herein, all individual members of the group and all combinations and sub-combinations possible of the group are intended to be individually included in the disclosure. Whenever a range is given in the specification, for example, a temperature range, a time range, or a composition range, all intermediate ranges and sub-ranges, as well as all individual values included in the ranges given are intended to be included in the disclosure.

As used herein, “comprising” is synonymous with “including,” “containing,” or “characterized by,” and is inclusive or open-ended and does not exclude additional, un-recited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed in the detailed description of the invention are used as terms of description and not of limitation, and there is no intention in the use of such terms and expressions of excluding any equivalents of the features shown and described, or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Disclosed herein is a new plant protein source termed, quinoa protein concentrate (“QPC”), having a protein content of at least about 50 wt % on a dry weight basis, and other key components contained in quinoa (Chenopodium quinoa Chenopodiaceae) grain and capable of isolation thereform. Despite the recent interest in quinoa in the food, paper, and cosmetic industries due to its unique starch properties and high lipid content compared to other cereals, quinoa as a plant protein source has not been explored. The inventors herein discovered an efficient process by which maximum amounts of quinoa protein, as well as other isolated components of commercial value, such as oil, fiber, and starch contained therein, can be obtained.

### Example 1

### Quinoa Protein Concentrate (QPC)

The process provides means to isolate individual components of nutritional and commercial value from quinoa grain, all of which are food- and pharmaceutical-grade. For example, quinoa oil, which is present at about 6-9% in unprocessed quinoa seed, can be obtained at a level above 80% from the initial solvent extraction or mechanical extraction (e.g., cold pressing or expeller pressing). Likewise, the starch level obtained from the process is above 80%. Quinoa fiber isolated from the process is at a level of at least 30%, or, alternatively can be isolated at a level of at least 50%. The steps indicated with an asterisk (*) in the associated figures are optional in isolating quinoa protein concentrate. In other words, one can obtain quinoa protein concentrate in the range of at least about 50% on a dry weight basis without carrying out the steps indicated as optional. Accordingly, the present invention provides a quinoa protein concentrate having at least about 50 wt %, specifically at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, or 99 wt % on a dry weight basis.

QPC isolated by the process can be used instead, of or in combination with, other plant proteins such as alfalfa proteins, grass proteins, soya proteins and rape proteins, etc., or animal proteins such as milk proteins and meat proteins in pet food and animal feed. QPC can also be used in processed foods, diet foods, health food or nutritional supplements, gluten free products, and as a substitute for wheat and other grains, milk, and eggs. QPC is also useful for nutritional purposes as a source of high quality protein in a wide variety of high-energy food and beverage products (protein bars, protein drinks, and nutritional beverages including meal replacement drinks).

QPC may be used in conventional applications of protein concentrates, such as protein fortification of processed foods, emulsification of oils, body formers in baked goods and foaming agents in products which entrap gas. QPC can also be used for a variety of functional effects that are associated with proteins, e.g., as a gelation aid in yogurts and pudding, as a water binder in meat and sausage, as a foaming or whipping aid in toppings and fillings, and as an emulsifier in ice cream, margarine and mayonnaise. In addition, QPC may be formed into protein fibers, useful in meat analogs, and may be used as an egg white substitute or extender in food products where egg white is used as a binder. Other uses of QPC are in edible films and capsules, biodegradable packaging, industrial and cosmetic applications, and in personal care products. QPC can replace all or a portion of the fat or cream in food products such as ice cream, yogurt, salad dressing, mayonnaise, cream, cream cheese, other cheeses, sour cream, sauces, icings, whipped toppings, frozen confections, milk, coffee whitener and spreads. QPC can be hydrolyzed to produce a variety of vegetarian flavors as in the case with hydrolyzed vegetable proteins from soy.

Preparation of QPC from Quinoa

Sample Preparation:

Quinoa grain was harvested and cleaned with sieves and shaking belts to remove stems, rocks, and debris, similar to the manner by which other grains are cleaned prior to processing. Optionally, quinoa can further be mechanically abraded, similar to rice polishing, to remove the outer pericarp (or hull) before further processing such as described below.

Fat Extraction:

Whole quinoa grain was flaked, similar to oat flakes, at ambient temperature, using flaker (Series No. 2188 size 18×12 HD, Ross Machine & Mill Supply, Oklahoma City, Okla.) with a roll gap of 0.051 mm, or similar art-known flaking equipment, with or without tempering to adjust the moisture content of the grain to achieve optimum results. Alternatively, whole quinoa grain may be comminuted (ground, cracked, crushed or milled, etc.) or a combination thereof, with or without tempering to adjust the moisture content of the grain to achieve optimum results.

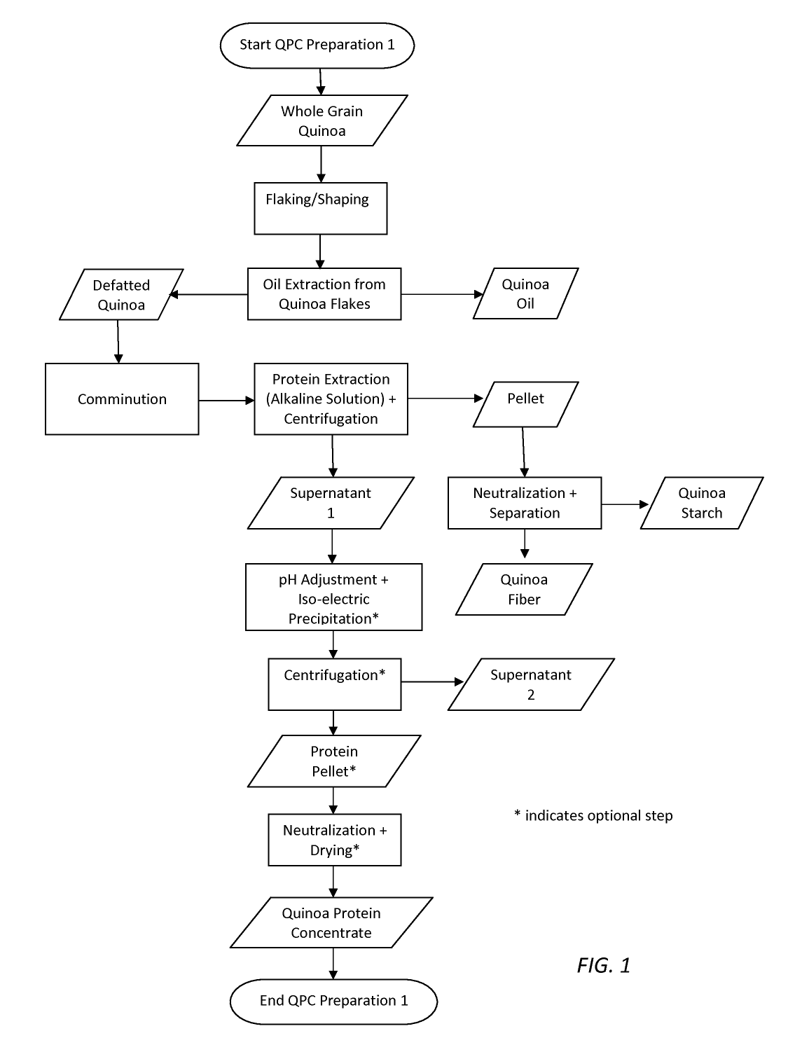

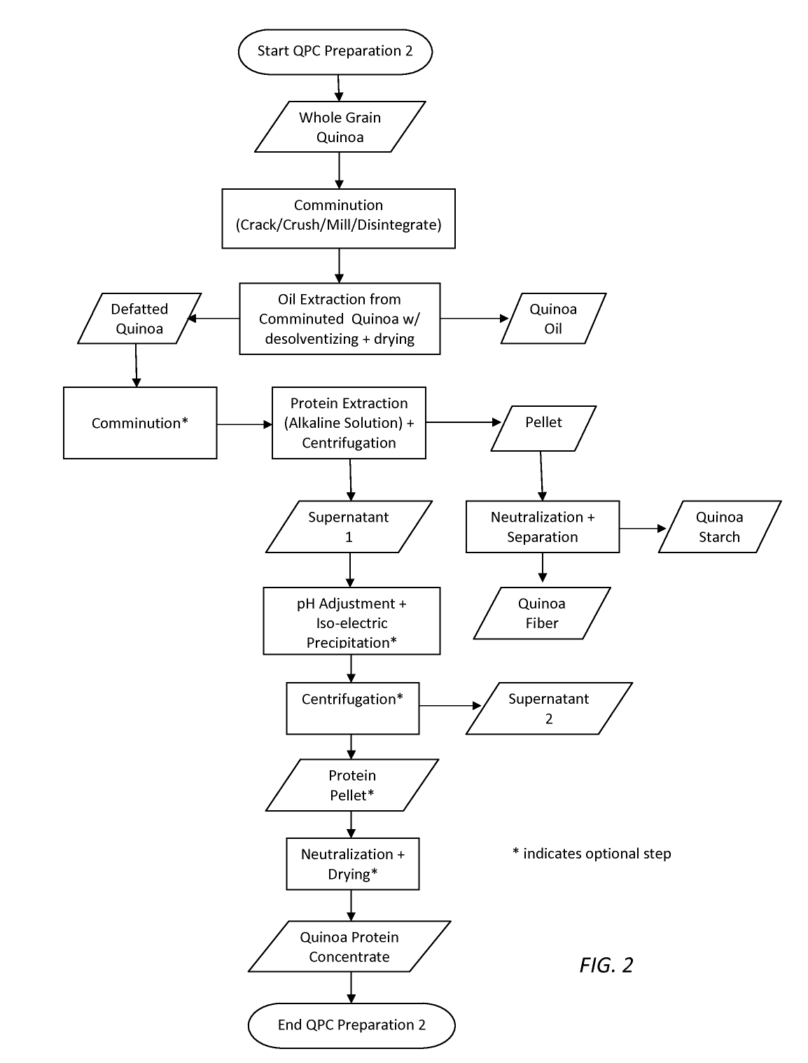

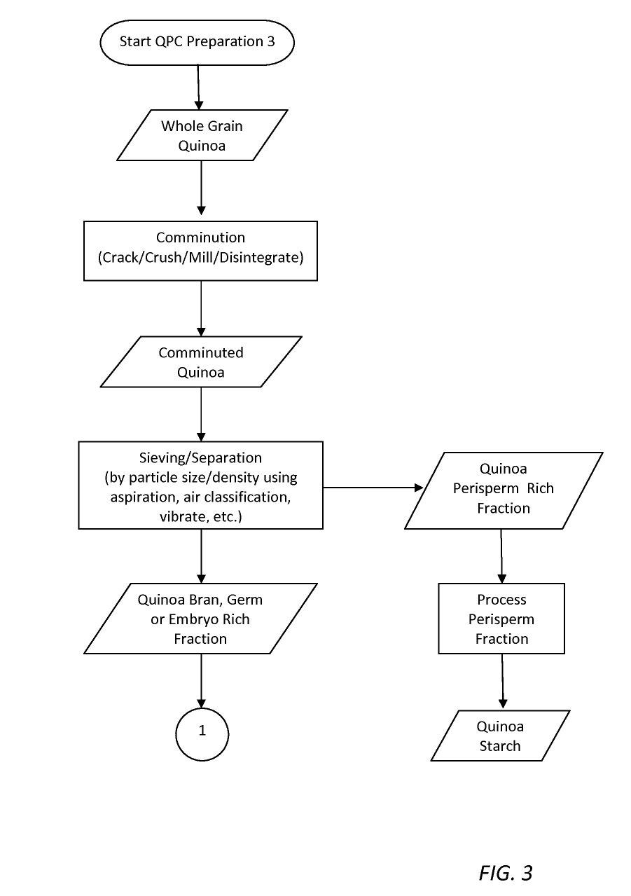

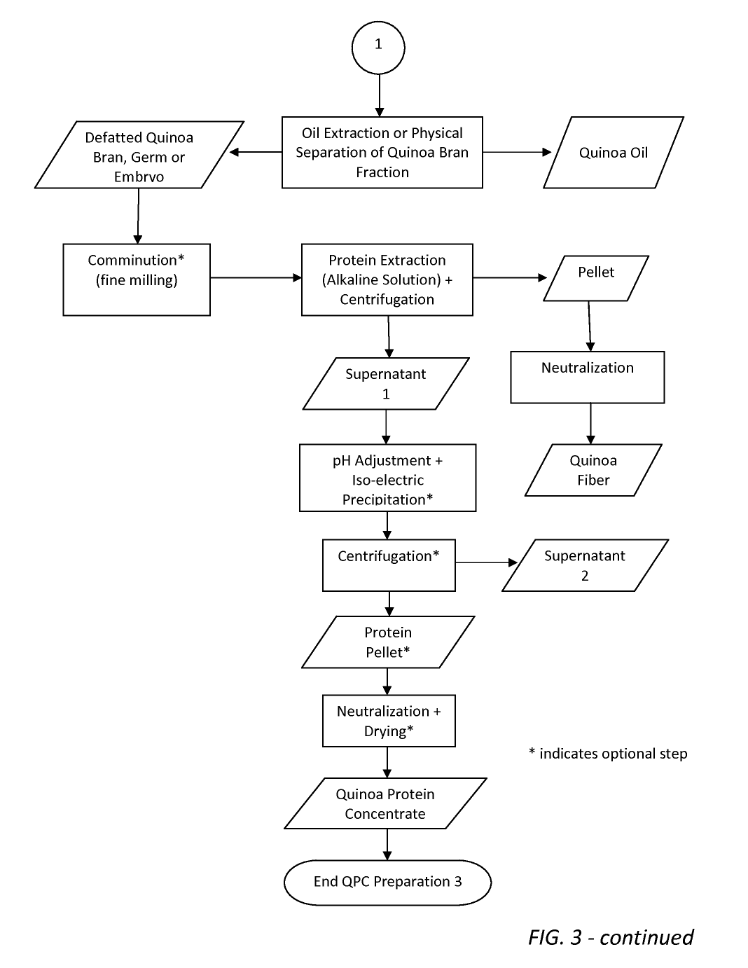

Quinoa oil was extracted from quinoa flakes with 1:1 w/v (quinoa:ethanol) using lab Model IV oil extractor, size 0.25 cu ft (Crown Iron Works, Roseville, Minn.). Quinoa oil was also extracted from quinoa flakes on a larger scale, using industry equipment, with 1:1 w/v (quinoa:ethanol) using Model IV oil extractor, size 1.9 cu ft (Crown Iron Works, Roseville, Minn.). Quinoa oil can be extracted from quinoa flakes (as in Scheme 1 which is outlined in FIG. 1), comminuted quinoa (as in Scheme 2 which is outlined in FIG. 2), or bran, germ or embryo rich fractions (as in Scheme 3 which is outlined in FIG. 3) using similar art-known oil extraction equipment. Other nonpolar solvents, such as hexane, methanol, acetone, and isopropyl alcohol, can also be used to extract oil.

The step of fat extraction, or “defatting” can be carried out at later steps, for example, after concentrating and drying the protein, if desired. Other methods of defatting can be used including supercritical liquid CO₂ extraction and mechanical pressing. The preferred ratio of quinoa to solvent is about 1:1 (w/v) and residence time in the extractor is 60 min, however, this ratio and residence time can be adjusted depending on the solvent and a given sample of quinoa. Quinoa oil micelle and solvent mixture was separated from the quinoa marque (the defatted material containing protein, starch, fiber etc.) using the oil extractor equipment. The solvent was recovered from the quinoa oil and the quinoa marque was desolventized and dried with mild heat, to prevent or minimize damage to protein and starch, using Down Draft Desolventizer-Toaster-Dryer-Cooler (Crown Iron Works, Roseville, Minn.). Solvent can be recovered from quinoa oil and quinoa marque using similar art-known oil desolventizer equipment. The oil was refined further by physical and/or caustic refining, similar to corn and soybean oil refining. Desolventizing and drying quinoa marque removes moisture and residual solvent and what remains is referred to as defatted quinoa.

Protein Extraction:

Ten grams of defatted quinoa was milled finely to about 100 microns or less, using a Lab Micro Mill, to yield defatted quinoa flour (also called oil seed meal). To extract protein, the defatted quinoa flour was suspended in 100 ml of 0.03 mol/l sodium hydroxide (any food grade base can be used) and stirred mechanically at ambient temperature for about 4 hours to maximize solubility of the protein. The pH of the suspension was about 10. The suspension mixture was centrifuged for 30 minutes at 6,000 g at about 0-10° C. using a lab centrifuge. The supernatant (“super 1”) containing protein was separated from the pellet (“pellet 1”) containing fiber, starch, and insoluble protein.

Quinoa protein was also extracted from defatted quinoa flour on a larger scale, using industry equipment. Defatted quinoa was milled finely to about 100 microns or less, using a Pin Mill, to yield defatted quinoa flour. To extract protein, the defatted quinoa flour was suspended in 0.03 mol/l sodium hydroxide and stirred mechanically at ambient temperature ranging for 2 to 5.5 hours to maximize solubility of the protein. The suspension mixture was centrifuged for 30 seconds at 3,500 g at ambient temperature using a decanter centrifuge and centrifuged for 60 seconds at 7,000 g at ambient temperature using a disc stack centrifuge. The supernatant (“super 1”) containing protein was separated from the pellet (“pellet 1”) containing fiber, starch, and insoluble protein. This separation can also be achieved using similar centrifuge equipment or hydrocyclone separators that are well-known in the art. Alternatively, quinoa protein can be extracted from quinoa bran, germ or embryo rich fractions (as in Scheme 3 which is outlined in FIG. 3).

The optimal ratio of the defatted quinoa flour to alkaline solution is 1:10 (w/v), however, this ratio can be adjusted, if necessary, and the molarity of the alkaline solution and defatted quinoa flour suspension can be adjusted to obtain a pH in the range of 8-12. The temperature is not critical for this step and can be readily modified. The length of the extraction should be adjusted to maximize protein recovery. In our hands, about 4 hours yielded most protein.

The pH of the super 1 was then adjusted to about 4.25 with hydrochloric acid (any food grade acidulant can be used) in order to precipitate the protein. The pH for this step can be in the range of 3-6.5. The pellet containing protein precipitates was separated by centrifugation. On a lab scale, the protein precipitates were centrifuged for 30 minutes at 13,000 g at about 0-10° C. The newly obtained pellet (“pellet 2”) can be used as a protein source as it is at this stage. Generally, the protein pellet is resuspended in a small volume of water (e.g., 1 g/10 ml H₂O), neutralized (˜pH 7) and freeze-dried. Alternatively, the protein precipitates were settled, the supernatant (“super 2”) was decanted, and the settled protein was neutralized (˜pH 7) and freeze-dried. On a larger scale, using industry equipment, the protein precipitates were centrifuged for 60 seconds at 7,000 g at ambient temperature using a disc stack centrifuge. The newly obtained pellet (“pellet 2”) can be used as a protein source as it is at this stage; however, the pellet may also be neutralized (˜pH 7) and spray-dried.

Alternatively, the protein does not have to be precipitated. The pH of the super 1 can be adjusted in the range of about 6 to 8, preferably about 7.0. Quinoa protein can be prepared from this neutralized protein fraction simply by drying or dewatering the protein using filtration followed by drying the protein.

The protein pellet can be separated using other means such as hydroclone separators or simply by letting the protein settle over time.

The product obtained at this stage typically contains about 90 wt % protein, on a dry weight basis, as determined by micro-Kjeldahl method or Dumas combustion method [American Association of Cereal Chemists: “Approved Methods of Analysis,” The Association, St. Paul, Minn., 2000]. Depending on the exact procedure used to obtain the protein concentrate from quinoa (referred herein as “quinoa protein concentrate”), the protein content ranges from about 50 wt % to at least about 90 wt %.

Starch and Fiber Extraction:

The pellet 1 obtained as above was resuspended in 100 ml of water on a lab scale. The suspension was neutralized and vacuum filtered through a series of wire mesh cloths, with select mesh sizes, in order to separate the starch from the material such as fiber and insoluble proteins. Alternatively, the pH of the suspension was adjusted to about 5.5 (the range for cellulase activity is 3 to 7) and the temperature was increased to about 50° C. (the range of cellulase activity is 25-70° C.). Carbohydrases, specifically cellulases, enzymes that catalyze the breakdown of cell walls, into glucose, cellobiose and higher glucose polymers, were added to the suspension. The pH and the temperature were maintained during the enzyme digestion for about 1 hour. The digest was neutralized and vacuum filtered through a series of wire mesh cloths in order to separate the starch from the partially digested fiber and insoluble proteins. The digestion step using cellulases improves the yield of quinoa starch. On a larger scale, using industry equipment, pellet 1 was resuspended in water, neutralized and sieved through a series of screens, with select mesh sizes, using a vibratory separator. This step can be carried out by equipment, such as cyclones, that are known in the art. The separated starch was spray dried. The separated fiber was spray dried. Alternatively, quinoa starch can be extracted from perisperm rich fractions and quinoa fiber can be extracted from quinoa bran, germ or embryo rich fractions (as in Scheme 3/FIG. 3). A Buhler Mill was used to separate the bran, germ or embryo rich fractions from the perisperm rich fraction.

Quinoa Oil:

Quinoa has potential to be a greater and more nutritional source of oil than oil produced from cereals crops (Fleming and Galwey 1995 supra). The oil content of quinoa is about 5.6%, with some varieties having lipid contents up to 9.5%. The yield of extractable vegetable oil per hectare could easily exceed that obtained from maize (80-400 kg/ha and 20-50 kg/ha, quinoa and maize, respectively) making quinoa a valuable new oil crop [Koziol (1990) In: Wahli, ed. Quinua: Hacia su Cultivo Comercial. Latinreco S A, Casilla, 17-110-6053. Quito, Ecuador, ppg. 137-159]. Quinoa oil is rich in unsaturated fatty acids. Although desirable nutritionally, unsaturated fatty acids are unstable to oxidation. However, quinoa oil is quite stable due to high levels of natural antioxidant vitamin E, 690-740 ppm α-tocopherol and 790-930 ppm γ-tocopherol. Although Koziol found concentrations fall to 450 and 230 ppm, respectively, after refining, 100-200 ppm is sufficient for optimal antioxidant activity of these isomers [Hudson and Ghavami (1984) Lebensm Wiss U Technol. 72: ppg. 82-85].

High lipid content compared with traditional cereals and essential fatty acid profile make quinoa a potential valuable oil crop. Quinoa oil is a rich source of essential fatty acids linoleic and linolenic, which constitute approximately 55-63% of the oil [Ruales and Nair (1993) Food Chemistry 48(2): ppg. 131-136; Fleming and Galwey (1995) supra], and make it similar to that of soya oil. In a comparison of fatty acids and triacylglycerol compositions, quinoa oil had the lowest saturate/unsaturate ratio compared with oils from five Amaranthus accessions, buckwheat, corn, ricebran, sesame, soybean and cottonseed [Jahaniaval, F et al. (2000) JAOCS 77(8): ppg. 847-852]. In addition, quinoa and soybean oils had the most favorable linoleic to linolenic acid ratio of the preceding oils.

Starch and Other Carbohydrates:

Studies on the physico-chemical characteristics of quinoa starch have been carried out by Wolf, M J et al. (1950) Cereal Chem. 27: ppg. 219-222; Scarpati de Briceno and Biceno (1982) In: Tercer Congreso Internacional de Cultivos Andinos. Ministerio de Asuntos Campesinos y Agropecuarios La Paz, Bolivia. 8-12(2): ppg. 69-77; Atwell, W A et al. (1983) Cereal Chem. 60(1): ppg. 9-11; Varriano-Marston and deFrancisco A (1984) Food Microstructure 3: ppg. 165-173; and Lorenz (1990) Starch/Stark 42(3): ppg. 81-86. The size of the starch granule and its amylose content are important factors in determining functional properties in food systems. Starch granules occur in the perisperm cells as compound granular aggregates and the individual starch granule is small and uniform with an average particle size of 1-2 μm [Atwell, et al (1983) supra], compared with that of maize and wheat, 1-23 ρm and 2-40 μm, respectively (Wolf, et al (1950) supra; Swinkels, J J M (1985) In: Van Beynum G M A and Roels J A, eds. Starch Conversion Technology. Dekker, New York. ppg. 15-46]. Although small starch granules have been shown to have reduced baking potential [Kulp, K (1973) Cereal Chem. 50: ppg. 666-679], the small size and uniformity of quinoa starch granules impart a smooth texture and mouth-feel. This attribute has gained considerable interest from food, paper, and cosmetic industries. Consequently, in 1989 European Patent Application No 89121654.1 was established for the manufacture of a carbohydrate-based cream substitute from quinoa starch. Small quinoa starch granules also have application as inexpensive filler in low density polyethylene films [Ahamed, N T et al. (1996) Carbohydrate Polymers 31(3): ppg. 157-160].

Atwell, et al (1982) supra performed an in-depth characterization of quinoa starch. Analysis indicates 11% amylose content, which is low in comparison to most cereal starches. It is comparable, however, to some varieties of rice, as reported by Williams, V R et al. (1958) J. Agric. Food Chem. 6: pg. 47. Lorenz (1990) supra, found that quinoa starch performs poorly in cake and bread baking due to its low amylose content and small starch granule. The researcher also found a higher swelling power of quinoa starch than that of barley, wheat, rice, amaranth, and potato, thus performing well as a thickening agent in fillings.

Free sugars in quinoa were evaluated to contain 4.55%, 2.41%, and 2.39%, glucose, fructose, and sucrose, respectively [Gonzalez, J A et al. (1989) Plant Foods for Human Nutr. 39: ppg. 331-337]. In the same study, the starch level was much lower than that reported by other authors [Ranhotra, G S et at (1993) Cereal Chem. 70(3): ppg. 303-305]. Consequently, due to high enzyme activity, starch levels will decrease and free sugars will increase upon grinding into flour.

### Example 2

### Isolation of the Perisperm Fraction from Quinoa Grain and Methods for Manufacturing Edible Quinoa Flour

In a second aspect the present invention provides methods of isolation of the perisperm fraction of the quinoa grain from the fraction containing the germ and further provides methods for manufacturing a edible quinoa flour having improved flavor, shelf-life, rheological, cooking and baking characteristics. Also provided are numerous uses for the edible quinoa flour produced according to the methods taught herein. Additional aspects derived from the method of manufacturing the edible quinoa flour, are the novel quinoa flour obtained by practicing the methods of the invention. As indicated above, this quinoa flour is distinctive in its improved flavor, shelf-life, rheological, cooking and baking characteristics.

A novel quinoa flour is prepared from quinoa (Chenopodium quinoa) grain using methods described herein. Methods of making quinoa flour have been described in International Publication No. W02005/058249 and US Patent Application 20070092629 titled “Quinoa protein concentrate, production, and functionality”, the contents of which are incorporated by reference. The quinoa flour of the instant invention has extended shelf-life and improved flavor, rheological, cooking and baking characteristics compared to today's commercially available quinoa flour ground from whole grain quinoa or partially treated or processed quinoa grain. One undesirable characteristic of previously available quinoa flour is its flavor, which is often a grassy flavor, or a bitter flavor, or an off-flavor from oxidation. The quinoa flour of the invention has reduced saponin content and reduced enzyme level and/or activity. The quinoa flour of the invention also has a reduced bitter flavor and a reduced grassy or off flavor. The method described herein for the manufacture of quinoa flour of the invention is also designed with consideration of both the physical structure of the quinoa grain and the demands of industry-scale production.

Considerable interest has been focused on the need to develop hypo-allergenic food sources. The quinoa flour of the invention contains hypo-allergenic proteins, as opposed to allergenic proteins from milk, egg, fish, crustacean shellfish, tree nuts, wheat, peanuts, soybeans, and gluten proteins from wheat, barley, rye, triticale, and oat. The quinoa flour contains proteins that are high in lysine and histidine, and methionine and cystine, which are often limiting in plant proteins of grains and legumes, respectively. Therefore, the quinoa flour is useful as food ingredients and supplements to provide nutrients as well as necessary functionality in a variety of food products.

The quinoa flour produced according to the methods of the invention has numerous applications. For example, the quinoa flour of the invention can be used in a wide variety of products including but not limited to bakery products, tortillas, crackers, granolas, toppings, batters and coatings, confections, breakfast cereal, instant and cook-up porridge, pudding or tapioca-like pudding, snack and extruded foods, frozen foods, beverages, milk-like beverages, ready-to-eat packaged foods, foods for infants and toddlers, nutrition bars, meat extenders, meat analogs, pastas, dough, sauces, seasoning and dry ingredient mixes and the like as a thickener, bulking agent, or flavor carrier. The quinoa flour of the invention is particularly useful as a one-to-one replacement for all purpose wheat flour in pancakes, pie crusts and cookies. The quinoa flour of the invention also finds ready application as a replacement, or partial replacement, of corn or rice or potato-based food products. This can be particularly important because the corn or rice or potato-based food products, while also hypo-allergenic, are either low in protein content or limited in essential amino acid, lysine when compared to the quinoa flour of the invention.

The quinoa flour produced according to the methods of the invention can also be used in milk-free and dairy-free, soy-free, nut-free, peanut-free, gluten-free, and egg-free food or cosmetic products intended for use in subjects who require less- or hypo-allergenic food products. Similarly, the quinoa flour can be used in plant-based, vegan, vegetarian, non-GMO and non-genetically engineered food or cosmetic products.

In addition, quinoa flour can serve as an ingredient in pet foods and animal feeds, such as cattle feed. The FDA has banned the use of animal protein in cattle feed as a preventative measure against bovine spongiform encephalopathy (i.e., BSE or mad cow disease) [DEPARTMENT OF HEALTH AND HUMAN SERVICES (2004), Food and Drug Administration, 21 CFR Parts 189 and 700, [Docket No. 2004N0081], RIN-0910-AF47, Use of Materials Derived From Cattle in Human Food and Cosmetics]. The quinoa flour can be used to provide a protein source in pet foods and animal feeds to replace the banned animal protein.

Overview of Method of Making Quinoa Flour:

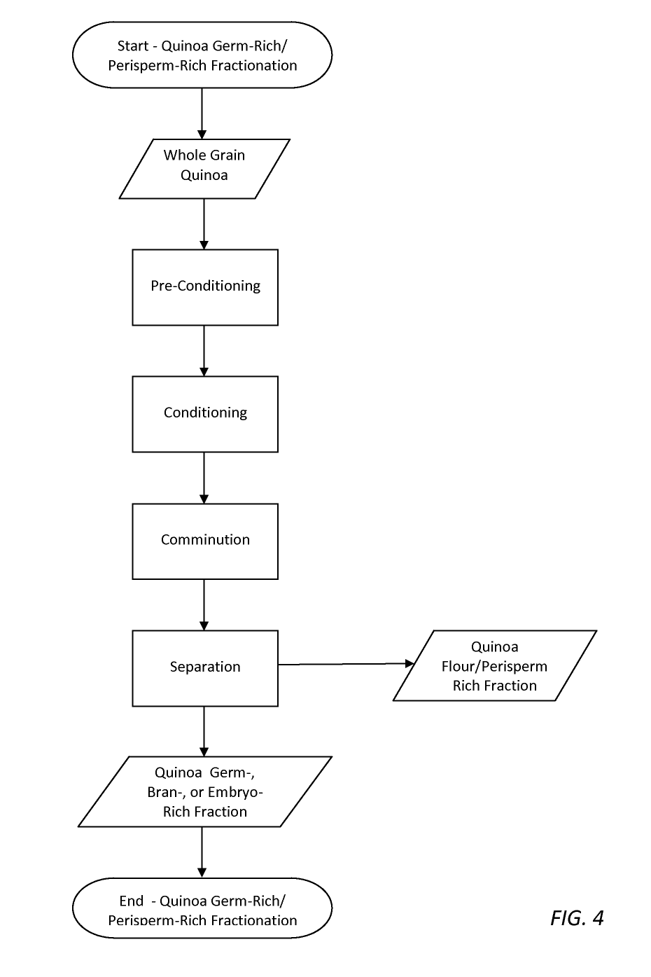

The present invention provides a non-obvious method for manufacturing quinoa flour. The method can be characterized by the steps of: 1) pre-conditioning the quinoa grain; 2) conditioning the pre-conditioned quinoa grain; 3) comminuting the conditioned quinoa grain; and 4) separating the fraction containing a majority of the germ from the fraction containing a majority of the perisperm. FIG. 4 illustrates the steps outlined above. When processing for quinoa flour (or the perisperm-rich fraction), the process also yields a valuable by-product in the quinoa germ-rich fraction (also referred to as the bran-rich fraction or embryo-rich fraction). The by-product has numerous valuable uses, some of which are addressed further in the examples below (i.e. Examples 4-6). The method of making quinoa flour yields quinoa flour with improved flavor, shelf-life, rheological, cooking and baking characteristics. The quinoa flour can be further treated to include particle size reduction, drying, tempering, bleaching, maturation, enrichment with nutrients, blending with quinoa germ to produce whole grain flour, fortification, agglomeration, pre-gelatinization, nixtamalization, cooking, and/or toasting if necessary. Prior to pre-conditioning, conditioning, or comminution the quinoa grain can be sorted by size, shape, or color prior to aid in the quality of the finished product.

Detailed Method of Isolating Perisperm Fraction and Manufacturing Edible Quinoa Flour:

Pre-Conditioning:

The term “pre-conditioning” is used herein to indicate a step of treatment to remove saponins in the quinoa grain. Saponins are concentrated in the pericarp of quinoa. Saponin removal can be achieved via mechanical abrasion or washing, and/or a combination of both. A preferred pre-conditioning includes abrasion followed by an initial quick wash with stiffing or agitation or spray or counter current extraction immediately followed by draining or centrifugation to minimize penetration of the water-soluble saponins into grain, followed by a second wash with stirring or agitation followed by draining or centrifugation. The number of washings can be adjusted in the range of one to ten washings, preferably about 2. The ratio of quinoa to water (w/v) can be adjusted to include ratios such as 0.1:1 or 0.5:1; 2:1; 3:1; 4:1; 5:1; 20:1 or similar ratios, preferably 1:1. Residence time of initial quick wash and secondary wash can be adjusted in the range of 30 seconds to 60 minutes depending on a given variety of quinoa; preferably 30 seconds to 2 minutes for the initial wash and 2 minutes to 10 minutes for one or more of the secondary washes, most preferably (about) 1 minute for initial quick wash and (about) 5 minutes for secondary wash. Alternatively, pre-conditioning can include polishing, peeling, aspiration, air classification, sieving, pneumatic pressure, vacuum, nixtamalization, rinsing, and/or solvent leaching. The pre-conditioning step described herein is preferably immediately followed by conditioning.

Conditioning:

The term “conditioning”, or “conditioned”, is used herein to indicate treatment to adjust the moisture content of the quinoa grain. Conditioning can be employed for the effect of tempering or regulating the moisture content. The moisture content can be adjusted by the addition or removal of water. A preferred conditioning technique includes drying grain or evaporating water immediately after pre-conditioning in the range of about 12 to 30% moisture content of grain, preferably about 13 or about 14%. Suitable drying methods include evaporation with vacuum or air circulation through grain at a temperature range of 0-300° C., preferably ambient temperature. A preferred drying method occurs in a rapid time to minimize microbiological growth and product deterioration and minimize penetration of water into the perisperm. The moisture of the quinoa can be allowed to equilibrate. However, it is preferable to control the residence time of conditioning to minimize penetration of water into the perisperm. Conditioning time is reduced compared to tempering times practiced in traditional milling. Alternatively, grain can be dried to include a range such as 5 to 11% moisture content, preferably about 10% and can be stored prior to conditioning. However, drying time and temperature should be controlled to minimize microbiological growth and deterioration. After storage, the moisture content can be adjusted in the range of about 11 to 30%, preferably about 13 or about 14%. Alternatively, conditioning can include but not limited to steeping, soaking, and/or or steaming. Alternative grain drying methods exist in the industry and literature and frequently use heat.

Comminuting:

The term “comminuting”, or “comminuted”, is used herein to indicate a step of treatment such as milling with break or corrugated rolls and/or reduction or smooth rolls. A preferred comminuting technique includes passing the conditioned grain through a motorized mill with 2 corrugated rollers with a gap setting of 0.025 inch, followed by passing the grain through 2 corrugated rollers with a gap setting of 0.015 inch, followed by passing the grain through 2 corrugated rollers with a gap setting of 0.010 inch, followed by passing the grain through 2 smooth rollers with a gap setting of about 0.008 inch or less. The number of times the grain material passes through rollers can be adjusted in the range of one to twelve, preferably about 4. The gap settings on the corrugated and smooth rollers can be adjusted to include such as 0.00005 to 0.10 inch or similar gap settings. Alternatively, comminuting can include polishing, abrasion, milling, pin milling, hammer milling, degerming, stone grinding, cracking, crushing, slicing, and/or flaking.

Separating:

The term “separating”, or “separation” or “separate”, is used herein to indicate a step of treatment such as sieving, whereby the germ-rich fraction is separated from the peri-sperm rich fraction. The preferred separation technique includes passing the comminuted grain material through a motorized sifter with a screen made from stainless steel woven wire cloth 18×18 mesh, 0.015″ wire diameter, followed by sifting the material that passes through the 18×18 mesh screen through a screen made from stainless steel woven wire cloth with 30×30 mesh, 0.0095″ wire diameter, collecting the material that is retained on both screens as the germ-, bran-, or embryo-rich fraction, and collecting the material that passes through the 30×30 mesh screen as the perisperm-rich fraction, or quinoa flour of the invention. Steps 1, 2, 3, and 4 (i.e. pre-conditioning, conditioning, comminuting and separating) practiced in this manner will produce a novel germ-rich fraction that is flattened or flaked similar to thin oat flakes and a powdery peri-sperm rich fraction, or quinoa flour of the invention. Alternatively, separation can occur prior to smooth rolling because embryo-rich fraction is lighter and the peri-sperm rich fraction is denser. Alternatively, the peri-sperm rich fraction does not have to go through smooth rollers and can remain coarse, such as that of semolina flour. The flour of the invention has reduced enzyme activity and increased shelf life because it is separated from embryo-rich fraction containing increased levels of metabolically active enzymes that can lead to product deterioration and off-flavors. Alternatively, the number of screens used in the sifter, the mesh size of the screens, the wire diameter, the micron openings, and the material of the screens can be adjusted. Alternatively, separation can include but not limited to aspiration, air classification, pneumatic pressure, vacuum, and/or vibration.

Quinoa starch can be readily obtained from quinoa flour of the invention by employing simple manipulations such as separation, extraction, or concentration. Methods of obtaining quinoa starch are presented in the examples below. It will be understood by those skilled in the art that the process disclosed herein can be operated with appropriate modifications and variations to obtain the afore-mentioned products. The process disclosed in this example is designed to separate the germ-rich fraction from the perisperm-rich fraction contained in quinoa grain, in a manner that enables one to obtain edible quinoa flour with novel characteristics as discussed above and illustrated in FIG. 4.

### Example 3

### Methods for Manufacturing Edible Quinoa Germ

In a third aspect the present invention provides methods of isolation of the germ fraction of the quinoa grain from the fraction containing the perisperm. Also provided are the quinoa germ produced according to the novel method, as well as numerous uses of the quinoa germ according to this method.

A novel germ-, bran-, or embryo-rich fraction of quinoa grain (“quinoa germ”) is prepared from quinoa (Chenopodium quinoa) grain using methods disclosed herein. Methods of processing quinoa grain are described in International Publication No. W02005/058249 and US Patent Application 20070092629 titled “Quinoa protein concentrate, production, and functionality”, the contents of which are incorporated by reference. The quinoa germ of the invention has a concentrated nutrient profile in comparison to quinoa grain, and acceptable flavor, cooking and baking characteristics. The quinoa germ of the invention also has a novel flattened or flake texture similar to thin oat flakes. There is currently no commercially-available quinoa germ today. The method described herein for the manufacture of quinoa germ of the invention is further designed with consideration of the physical structure of quinoa grain and for industry-scale production.

Considerable interest has been focused on the need to develop hypo-allergenic food sources, with particular interest in hypo-allergenic proteins. The quinoa germ of the invention contains hypo-allergenic proteins, as opposed to allergenic proteins from milk, egg, fish, crustacean shellfish, tree nuts, wheat, peanuts, soybeans, and gluten proteins from wheat, barley, rye, triticale, and oat. The quinoa germ contains proteins that are high in lysine and histidine, and methionine and cystine, which are often limiting in plant proteins of grains and legumes, respectively. Therefore, the quinoa germ is useful as food ingredients and supplements to provide nutrients as well as necessary functionality in a variety of food products.

The quinoa germ produced according to the methods of the invention has numerous applications. For example, the quinoa germ of the invention can be used in a wide variety of products including bakery products, tortillas, crackers, granolas, toppings, batters and coatings, confections, breakfast cereal, instant and cook-up porridge, pudding or tapioca-like pudding, snack and extruded foods, frozen foods, beverages, milk-like beverages, ready-to-eat packaged foods, foods for infants and toddlers, nutrition bars, meat extenders, meat analogs, pastas, dough, sauces, seasoning and dry ingredient mixes and the like as a thickener, bulking agent, or flavor carrier. The quinoa germ of the invention is particularly useful as a nutritional supplement or addition to bakery products and nutrition bars. The quinoa germ of the invention is particularly useful in the production of vegan, milk-like, beverages. The quinoa germ of the invention is also useful as a nutritional supplement. The quinoa germ of the invention also finds ready application as a replacement, or partial replacement, of corn- or rice- or potato-based food products, which are also hypo-allergenic, but are either low in protein content or limited in essential amino acid, lysine.

The quinoa germ produced according to the invention can be used in milk-free and dairy-free, soy-free, nut-free, peanut-free, gluten-free, and eggfree food or cosmetic products intended for use in subjects who require less- or hypo-allergenic food products. The quinoa germ can be used in plant-based, vegan, vegetarian, non-GMO and non-genetically engineered food or cosmetic products. In addition, quinoa germ can serve as an ingredient in pet foods and animal feeds, such as cattle feed, because the FDA banned the use of animal protein in cattle feed as a preventative measure against bovine spongiform encephalopathy (i.e., BSE or mad cow disease) [DEPARTMENT OF HEALTH AND HUMAN SERVICES (2004), Food and Drug Administration, 21 CFR Parts 189 and 700, [Docket No. 2004N0081], RIN-0910-AF47, Use of Materials Derived From Cattle in Human Food and Cosmetics].

Overview of Method of Making Quinoa Germ:

The present invention provides a non-obvious method for manufacturing quinoa germ. The method can be characterized by the steps of: 1) pre-conditioning quinoa grain; 2) conditioning the quinoa grain; 3) comminuting the quinoa grain; 4) separating the fraction containing a majority of the germ from the fraction containing a majority of the perisperm. FIG. 4 illustrates the steps outlined above. When processing for quinoa germ (i.e. germ-rich, bran-rich fraction or embryo-rich fraction or the perisperm-rich fraction), the process also yields a valuable by-product in the quinoa perisperm-rich fraction. The by-product has numerous valuable uses, including use as quinoa flour (Example 2) and quinoa starch (Example 7). The method of making quinoa germ yields a quinoa germ with concentrated nutrient profile, acceptable flavor, cooking and baking characteristics. The quinoa germ can be toasted for a different flavor and to extend its shelf-life as a food ingredient or refrigerated or frozen in its raw form to extend the shelf-life. The quinoa germ can be further treated to include particle size reduction, drying, tempering, bleaching, maturation, enrichment with nutrients, blending with quinoa flour to produce whole grain flour, fortification, agglomeration, pre-gelatinization, nixtamalization, cooking, and/or toasting if necessary. Prior to pre-conditioning, conditioning, or comminution the quinoa grain can be sorted by size, shape, or color to aid in quality of finished products.

Detailed Method of Isolating Germ Fraction and Manufacturing Edible Quinoa Flour:

Pre-Conditioning:

The term “pre-conditioning” is used herein to indicate a step of treatment to remove saponins in the quinoa grain. Saponins are concentrated in the pericarp of quinoa. Saponin removal can be achieved via mechanical abrasion or washing, and/or a combination of both. A preferred pre-conditioning includes abrasion followed by an initial quick wash with stiffing or agitation or spray or counter current extraction immediately followed by draining or centrifugation to minimize penetration of the water-soluble saponins into seed coat, followed by a second wash with stiffing or agitation followed by draining or centrifugation. The number of washings can be adjusted in the range of one to ten washings; preferably about 2. The ratio of quinoa to water (w/v) can be adjusted to include ratios such as 0.1:1 or 0.5:1; 2:1; 3:1; 4:1; 5:1; 20:1 or similar ratios, preferably 1:1. Residence time of initial quick wash and secondary wash can be adjusted in the range of 30 seconds to 60 minutes depending on a given variety of quinoa; preferably 1 minute for initial quick wash and 5 minutes for secondary wash. Alternatively, pre-conditioning can include polishing, peeling, aspiration, air classification, sieving, pneumatic pressure, vacuum, nixtamalization, rinsing, and/or solvent leaching. The pre-conditioning step described herein is preferably immediately followed by conditioning.

Conditioning:

The term “conditioning”, or “conditioned”, is used herein to indicate a step of treatment such as tempering or regulating the moisture content. The moisture content can be adjusted by the addition or removal of water. A preferred conditioning technique includes drying grain or evaporating water immediately after pre-conditioning in the range of about 12 to 30% moisture content of grain, preferably about 13 or about 14%. A preferred drying method includes evaporation with vacuum or air circulation through grain at a temperature range of 0-300° C., preferably ambient temperature. The drying method should occur in a rapid time to minimize microbiological growth and product deterioration, while minimizing penetration of water into the perisperm. The moisture of the quinoa can be allowed to equilibrate, however, it is preferable to control the residence time of conditioning to minimize penetration of water into the perisperm. Conditioning time is reduced compared to tempering times practiced in traditional milling. Alternatively, grain can be dried to include a range such as 5 to 11% moisture content, preferably about 10% and can be stored prior to conditioning, however, drying time and temperature should be controlled to minimize microbiological growth and deterioration. After storage, the moisture content can be adjusted in the range of about 11 to 30%, preferably about 13 or about 14%. Alternatively, conditioning can include steeping, soaking, and/or or steaming. Alternative grain drying methods exist in the industry and literature and frequently use heat.

Comminuting:

The term “comminuting”, or “comminuted”, is used herein to indicate a step of treatment such as milling with break or corrugated rolls and/or reduction or smooth rolls. A preferred comminuting technique includes passing the conditioned grain through a motorized mill with 2 corrugated rollers with a gap setting of 0.025 inch, followed by passing the grain through 2 corrugated rollers with a gap setting of 0.015 inch, followed by passing the grain through 2 corrugated rollers with a gap setting of 0.010 inch, followed by passing the grain through 2 smooth rollers with a gap setting of about 0.008 inch or less. The number of times the grain material passes through rollers can be adjusted in the range of one to twelve, preferably about 4. The gap settings on the corrugated and smooth rollers can be adjusted to include such as 0.00005 to 0.10 inch or similar gap settings. Alternatively, comminuting can include polishing, abrasion, milling, pin milling, hammer milling, degerming, stone grinding, cracking, crushing, slicing, and/or flaking.

Separating:

The term “separating”, or “separation” or “separate”, is used herein to indicate a step of treatment such as sieving, whereby the germ-rich fraction is separated from the peri-sperm rich fraction. The preferred separation technique includes passing the comminuted grain material through a motorized sifter with a screen made from stainless steel woven wire cloth 18×18 mesh, 0.015″ wire diameter, followed by sifting the material that passes through the 18×18 mesh screen through a screen made from stainless steel woven wire cloth with 30×30 mesh, 0.0095″ wire diameter, collecting the material that is retained on both screens as the quinoa germ-, bran-, or embryo-rich fraction of the invention, and collecting the material that passes through the 30×30 mesh screen as the perisperm-rich fraction, or quinoa flour. Steps 1, 2, 3, and 4 (i.e. pre-conditioning, conditioning, comminuting and separating) practiced in this manner will produce a novel germ-rich fraction that is flattened or flaked similar to thin oat flakes and a powdery peri-sperm rich fraction, or quinoa flour. Alternatively, separation can occur prior to smooth rolling because embryo-rich fraction is lighter and the peri-sperm rich fraction is denser. Alternatively, both the embryo-rich and peri-sperm rich fractions do not have to go through smooth rollers and can remain coarse. The embryo-rich fraction contains increased levels of metabolically active enzymes that can lead to product deterioration and off-flavors. Alternatively, the number of screens used in the sifter, the mesh size of the screens, the wire diameter, the micron openings, and the material of the screens can be adjusted. Alternatively, separation can include but not limited to aspiration, air classification, pneumatic pressure, vacuum, and/or vibration.

Quinoa protein, oil, and fiber can be obtained from the quinoa germ of the invention by employing methods discussed below, such as in Examples 4 through 6. Isolation of quinoa protein, oil, and fiber can also be made employing the techniques herein in conjunction with the methods discussed in International Publication No. WO2005/058249 and US Patent Application 20070092629 titled “Quinoa protein concentrate, production, and functionality”. It will be understood by those skilled in the art that the process disclosed herein can be operated with appropriate modifications and variations to obtain the aforementioned products. The process disclosed herein is designed to separate the germ-rich fraction from the perisperm-rich fraction contained in quinoa grain, in a manner that enables one to obtain edible quinoa germ with novel characteristics as discussed above and illustrated in FIG. 4.

### Example 4

### Methods for Manufacturing Edible Quinoa Oil and Uses Thereof

In a fourth aspect the present invention provides methods for manufacturing edible quinoa oil from the germ fraction of the quinoa grain, the quinoa oil produced by the method, and uses of the edible quinoa oil produced according to the methods of the invention.

Quinoa has potential to be a greater and more nutritional source of oil than oil produced from cereals crops (Fleming and Galwey 1995 supra). The oil content of quinoa is about 5.6%, with some varieties having lipid contents up to 9.5%. The yield of extractable vegetable oil per hectare could easily exceed that obtained from maize (80-400 kg/ha and 20-50 kg/ha, quinoa and maize, respectively) making quinoa a valuable new oil crop [Koziol (1990) In: Wahli, ed. Quinua: Hacia su Cultivo Comercial. Latinreco S A, Casilla, 17-110-6053. Quito, Ecuador, ppg. 137-159]. Quinoa oil is rich in unsaturated fatty acids. Although desirable nutritionally, unsaturated fatty acids are unstable to oxidation. However, quinoa oil is quite stable due to high levels of natural antioxidant vitamin E, 690-740 ppm α-tocopherol and 790-930 ppm γ-tocopherol. Although Koziol found concentrations fall to 450 and 230 ppm, respectively, after refining, 100-200 ppm is sufficient for optimal antioxidant activity of these isomers [Hudson and Ghavami (1984) Lebensm Wiss U Technlol. 72: ppg. 82-85].

High lipid content compared with traditional cereals and essential fatty acid profile make quinoa a potential valuable oil crop. Quinoa oil is a rich source of essential fatty acids linoleic and linolenic, which constitute approximately 55-63% of the oil [Ruales and Nair (1993) Food Chemistry 48(2): ppg. 131-136; Fleming and Galwey (1995) supra], and make it similar to that of soya oil. In a comparison of fatty acids and triacylglycerol compositions, quinoa oil had the lowest saturate/unsaturate ratio compared with oils from five Amaranthus accessions, buckwheat, corn, ricebran, sesame, soybean and cottonseed [Jahaniaval, F et al. (2000) JAOCS 77(8): ppg. 847-852]. In addition, quinoa and soybean oils had the most favorable linoleic to linolenic acid ratio of the preceding oils.

A novel, edible quinoa oil (or lipid-rich fraction) is prepared from the germ-, bran-, or embryo-rich fraction of quinoa grain (“quinoa germ”) from quinoa (Chenopodium quinoa) grain using methods disclosed herein. There is no commercially-available, edible quinoa oil today. The method described herein for the manufacture of quinoa oil of the invention is designed with consideration of the physical structure of quinoa grain and resultant oil, as well as for industry-scale production of the edible quinoa oil. The method described herein is designed to use the germ-, bran-, or embryo-rich fraction of quinoa grain as the starting material for the manufacture of quinoa oil in order to maximize cost-efficient production versus producing oil from whole quinoa grain as the starting material. Thus, the perisperm-rich fraction remains to be additionally utilized when preparing oil from the germ-rich fraction.

The quinoa oil of the invention can be manufactured from non-genetically engineered quinoa grain and therefore is particularly useful in natural and/or organic labeled products. The quinoa oil of the invention, while it may contain residual hypo-allergenic proteins, does not contain allergenic proteins such as those found in products derived from tree nuts, peanuts, and soybeans. It is therefore particularly useful in foods designed to replace edible oil containing residual allergenic proteins. The quinoa oil contains a high level of unsaturated to saturated fatty acids, vitamin E, and phospholipids. Therefore, the quinoa oil is useful as a food ingredient and dietary supplement to provide essential fatty acids and antioxidants as well as necessary functionality in a variety of food products and pet food products. The edible oil (or lipid-rich fraction) of quinoa of the invention is particularly useful as a source of hypo-allergenic food grade lecithin and squalene.

The quinoa oil produced according to the methods of the invention has numerous applications. For example, the quinoa oil of the invention can be used in a wide variety of products including but not limited to bakery products, tortillas, crackers, granolas, toppings, batters and coatings, confections, breakfast cereal, instant and cook-up porridge, pudding or tapioca-like pudding, yogurt, ice-cream, cheese, cream preparations, coffee whiteners and non-dairy creamers, whipped toppings, snack and extruded foods, frozen foods, beverages, milk-like beverages, powdered drink beverages, ready-to-eat packaged foods, fruit preparations, salad dressing, cooking oil, mayonnaise, shortening, margarine, foods for infants and toddlers, infant formula, nutrition bars, meat extenders, meat and seafood analogs, emulsified meats, canned meats, whole muscle and coarsely chopped meat products, seafood and poultry products, pastas, dough, candy and confections, sauces, seasoning and dry ingredient mixes. Additional uses in food include but not limited to viscosity control, to improve mouth-feel and texture, as frying oil for fried foods, as a processing aid, encapsulation, in production of low-glycemic and low-carb foods. Additional non-food uses include but not limited to personal care products, soaps, candles, hair spray, conditioners, shampoo, mousses, lotions, sunscreens and skin care preparations, cosmetics and nutraceuticals, encapsulation, cleansers, bio-fuels, paints, and anti-foaming agents.

The quinoa oil of the invention is particularly useful as a nutritional dietary supplement, edible cooking or salad oil. The quinoa oil of the invention is also particularly useful as a replacement for genetically engineered soybean or canola oil.

The quinoa oil can be used in milk-free and dairy-free, soy-free, nut-free, peanut-free, gluten-free, and egg-free food or cosmetic products intended for use in subjects who require less- or hypo-allergenic food products. The quinoa oil can be used in plant-based, vegan, low-glycemic, low-carb, vegetarian, non-GMO and non-genetically engineered food or cosmetic products. In addition, quinoa oil can serve as a nutritious ingredient in pet foods and animal feeds, such as cattle feed, since the FDA banned the use of animal protein in cattle feed as a preventative measure against bovine spongiform encephalopathy (i.e., BSE or mad cow disease) [DEPARTMENT OF HEALTH AND HUMAN SERVICES (2004), Food and Drug Administration, 21 CFR Parts 189 and 700, [Docket No. 2004N-0081], RIN-091 0-AF47, Use of Materials Derived From Cattle in Human Food and Cosmetics].

Overview of Method of Manufacturing Edible Quinoa Oil:

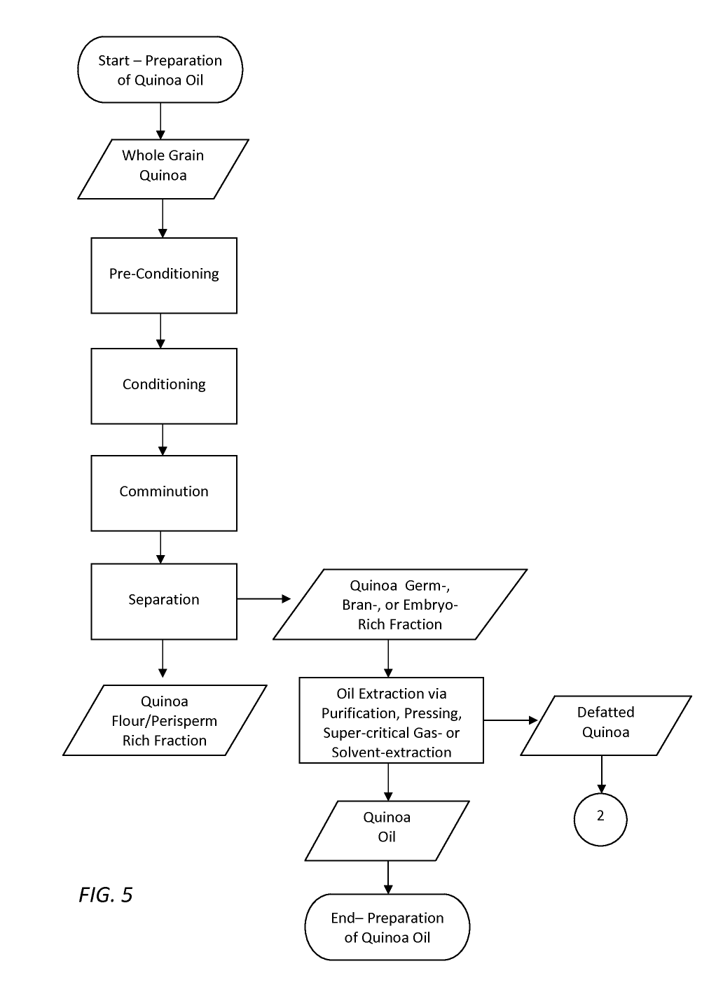

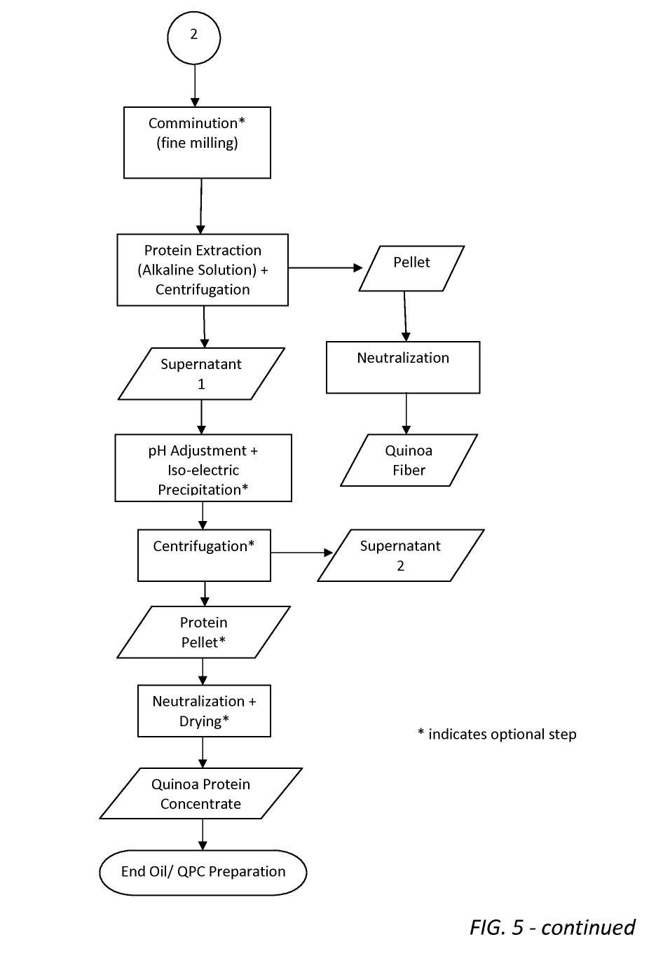

The present invention provides a non-obvious method for manufacturing edible quinoa oil. The method can be characterized by the steps of: 1) pre-conditioning the quinoa grain; 2) conditioning the quinoa grain; 3) comminuting the quinoa grain; 4) separating the fraction containing a majority of the germ from the fraction containing a majority of the perisperm; and 5) oil extraction from the germ fraction. Thus, a quinoa germ that is obtained as in Example 3, above, can be used as the starting material for the oil extraction, which can be accomplished via purification, mechanical pressing, supercritical gas-, or solvent-extraction. FIG. 5 illustrates the steps outlined above. The quinoa oil can be further treated by refining, deodorizing, bleaching, enzyme modification, and/or chemical hydrolysis if necessary. Quinoa grain can be sorted by size, shape, or color prior to pre-conditioning, conditioning, or comminution to aid in the quality of the finished product.

Detailed Method of Manufacturing Edible Quinoa Oil:

Pre-Conditioning:

The term “pre-conditioning” is used herein to indicate a step of treatment to remove saponins in the quinoa grain. Saponins are concentrated in the pericarp of quinoa. Saponin removal can be achieved via mechanical abrasion or washing, and/or a combination of both. Pre-conditioning of quinoa grain is described more fully above with respect to quinoa germ as in Example 3, above.

Conditioning:

The term “conditioning”, or “conditioned”, is used herein to indicate a step of treatment such as tempering or regulating the moisture content. The moisture content can be adjusted by the addition or removal of water. A preferred conditioning technique includes drying grain or evaporating water immediately after pre-conditioning in the range of about 12 to 30% moisture content of grain, preferably about 13 or about 14%. A preferred drying method includes evaporation with vacuum or air circulation through grain at a temperature range of 0-300° C., preferably ambient temperature. Conditioning of pre-conditioned quinoa grain is described more fully above with respect to quinoa germ as in Example 3, above.

Comminuting:

The term “comminuting”, or “comminuted”, is used herein to indicate a step of treatment such as milling with break or corrugated rolls and/or reduction or smooth rolls. A preferred comminuting technique includes passing the conditioned grain through a motorized mill with 2 corrugated rollers with a gap setting of 0.025 inch, followed by passing the grain through 2 corrugated rollers with a gap setting of 0.015 inch, followed by passing the grain through 2 corrugated rollers with a gap setting of 0.010 inch, followed by passing the grain through 2 smooth rollers with a gap setting of about 0.008 inch or less. Comminuting of conditioned quinoa grain is described more fully above with respect to quinoa germ as in Example 3, above.

Separating:

The term “separating”, or “separation” or “separate”, is used herein to indicate a step of treatment such as sieving, whereby the germ-rich fraction is separated from the peri-sperm rich fraction. The preferred separation technique includes passing the comminuted grain material through a motorized sifter with a screen made from stainless steel woven wire cloth 18×18 mesh, 0.015″ wire diameter, followed by sifting the material that passes through the 18×18 mesh screen through a screen made from stainless steel woven wire cloth with 30×30 mesh, 0.0095″ wire diameter, collecting the material that is retained on both screens as the quinoa germ-, bran-, or embryo-rich fraction of the invention, and collecting the material that passes through the 30×30 mesh screen as the perisperm-rich fraction, or quinoa flour. Separating the comminuted quinoa is described more fully above with respect to quinoa germ as in Example 3, above.

Oil Extraction:

Quinoa oil of the invention can be readily obtained from quinoa germ by employing manipulations such as purification, mechanical pressing, supercritical gas-, or solvent-extraction. Oil extraction is discussed in more detail above in Example 1. It will be understood by those skilled in the art that the process disclosed herein can be operated with appropriate modifications and variations to obtain the afore-mentioned products. The process disclosed herein is designed to separate the germ-rich fraction from the perisperm-rich fraction contained in quinoa grain, in a manner that enables one to obtain edible quinoa oil from the germ-rich fraction as the starting material.

### Example 5

### Methods for Manufacturing Quinoa Protein Concentrate

In a fifth aspect the present invention provides methods of manufacturing a quinoa protein concentrate (or quinoa protein isolate). Also provided are the quinoa protein produced according to the novel method, as well as numerous uses of the quinoa protein produced according to this method.

A novel, quinoa protein concentrate is prepared from the germ-, bran-, or embryo-rich fraction of quinoa grain (“quinoa germ”) from quinoa (Chenopodium quinoa) grain using methods disclosed herein. There is no commercially-available quinoa protein concentrate today. The method described herein for the manufacture of quinoa protein concentrate of the invention is designed with consideration of the physical structure of quinoa grain and for industry-scale production of edible protein concentrate. The method is designed to use the germ-, bran-, or embryo-rich fraction of quinoa grain as the starting material for the manufacture of quinoa protein concentrate in order to maximize cost-efficient production versus producing protein concentrate from whole quinoa grain as the starting material. Additional methods of processing quinoa grain are described in International Publication No. WO2005/058249 and US Patent Application 20070092629 entitled “Quinoa protein concentrate, production, and functionality”, the contents of which are incorporated by reference.

The quinoa protein concentrate of the invention can be manufactured from non-genetically engineered quinoa grain and therefore is particularly useful in natural and/or organic labeled products. The quinoa protein concentrate of the invention contains hypo-allergenic proteins, as opposed to allergenic proteins from milk, egg, fish, crustacean shellfish, tree nuts, wheat, peanuts, soybeans, and gluten proteins from wheat, barley, rye, triticale, and oat and therefore is particularly useful in foods designed to replace allergenic proteins. The quinoa protein concentrate contains proteins that are high in lysine and histidine, and methionine and cystine, which are often limiting in plant proteins of grains and legumes, respectively. Therefore, the quinoa protein concentrate is useful as food ingredients and dietary supplements to provide essential amino acids as well as necessary functionality in a variety of food products and pet food products.

The quinoa protein concentrate produced according to the methods of the invention has numerous applications. For example, the quinoa protein concentrate of the invention can be used in a wide variety of products including but not limited to bakery products, tortillas, crackers, granolas, toppings, batters and coatings, confections, breakfast cereal, instant and cook-up porridge, pudding or tapioca-like pudding, yogurt, ice-cream, cheese, cream preparations, coffee whiteners and non-dairy creamers, whipped toppings, snack and extruded foods, frozen foods, beverages, milk-like beverages, powdered drink beverages, ready-to-eat packaged foods, fruit preparations, salad dressing, foods for infants and toddlers, infant formula, nutrition bars, meat extenders, meat and seafood analogs, emulsified meats, canned meats, whole muscle and coarsely chopped meat products, seafood and poultry products, pastas, dough, candy and confections, sauces, seasoning and dry ingredient mixes. Additional uses in food include but are not limited to binders, emulsifiers, stabilizers, foaming agents, thickeners, viscosity control, fermentation aids and clarifiers, to improve mouthfeel and texture, coating agents for fried and baked foods, to reduce the caloric content or in the production of low-glycemic, and low-carb foods. Additional non-food uses include but not limited to encapsulation, adhesives, films, releasing agents, packaging, personal care products, hair spray, conditioners, shampoo, mousses, lotions, sunscreens and skin care preparations, and cosmetics and nutraceuticals, cleansers, bio-fuel, inks, leather substitutes, paints, paper, plastics, textile fibers, foaming agents.

The quinoa protein concentrate of the invention is particularly useful as an independent nutritional supplement or addition to nutrition bars, meal replacement bars, athlete-, sport-, and performance-enhancing nutritional foods, protein-rich energy bars and protein-rich beverages. The quinoa protein concentrate of the invention is particularly useful in the production of vegan, milk-like, beverages, infant formula, medicinal foods, and medicinal beverages (such as those in immuno-compromised disease states and for elderly). The quinoa protein concentrate of the invention is particularly useful as a replacement or partial replacement of corn- or rice or potato-based food products, which are also hypo-allergenic, but are either low in protein content or limited in the essential amino acid, lysine. The quinoa protein concentrate of the invention is particularly useful as an encapsulating agent, carrier, capsule and/or tablet agent in the preparation of food flavors, cosmetic fragrances and pharmaceuticals. The quinoa protein concentrate of the invention is particularly useful as a replacement for genetically engineered soybean protein, or as an ingredient or starting material for the manufacture of protein-based ingredients. The quinoa protein concentrate of the invention can be used as a source of non-genetically engineered starting material for the manufacture of amino acids, hydrolyzed vegetable protein, and textured protein.

The quinoa protein concentrate can be used in milk-free and dairy-free, soy-free, nut-free, peanut-free, gluten-free, and egg-free food or cosmetic products intended for use in subjects who require less- or hypo-allergenic food products. The quinoa protein concentrate can be used in plant-based, vegan, low-glycemic, low-carb, vegetarian, non-GMO and non-genetically engineered food or cosmetic products. In addition, quinoa protein concentrate can serve as a nutritious ingredient in pet foods and animal feeds, such as cattle feed, since the FDA banned the use of animal protein in cattle feed as a preventative measure against bovine spongiform encephalopathy (i.e. BSE or mad cow disease) [DEPARTMENT OF HEALTH AND HUMAN SERVICES (2004), Food and Drug Administration, 21 CFR Parts 189 and 700, [Docket No. 2004N-0081], RIN-0910-AF47, Use of Materials Derived From Cattle in Human Food and Cosmetics].

Overview of Method of Manufacturing Quinoa Protein Concentrate:

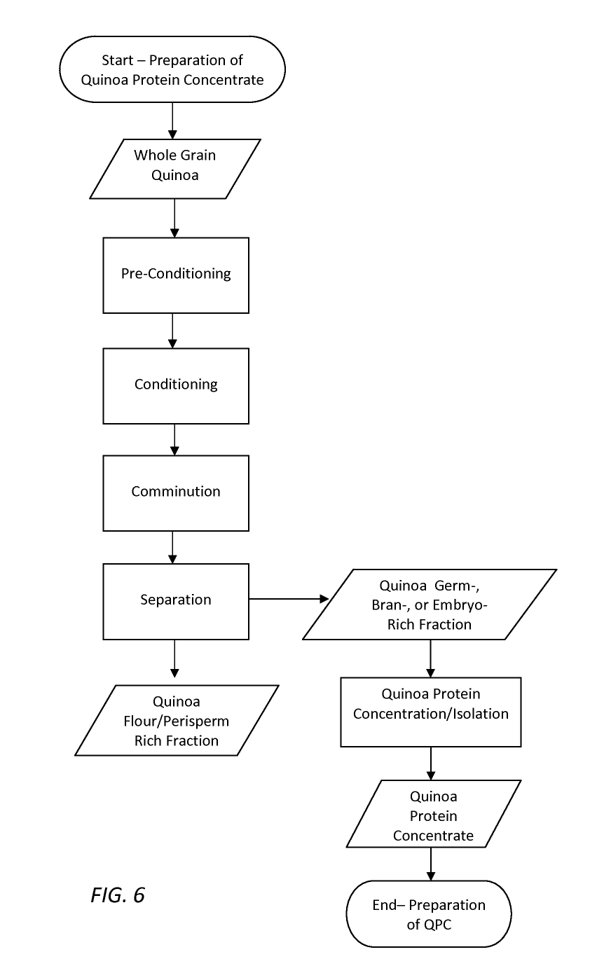

The present invention provides a non-obvious method for manufacturing quinoa protein concentrate. The method can be characterized by the steps of: 1) pre-conditioning the quinoa grain: 2) conditioning the quinoa grain: 3) comminuting the quinoa grain; 4) separating the fraction containing a majority of the germ from the fraction containing a majority of the perisperm; and 5) concentrating the resulting quinoa protein from the germ fraction. Thus, a quinoa germ that is obtained as in Example 3, above, can be used as the starting material for the for the protein concentrate. The protein may then be concentrated via purification, extraction, iso-electric precipitation, or other concentration methods. FIG. 6 illustrates the steps outlined above. The quinoa protein concentrate can be further treated by particle size reduction, drying, agglomeration, enzyme modification, and/or chemical hydrolysis if necessary. Quinoa grain can be sorted by size, shape, or color prior to pre-conditioning, conditioning, or comminution to aid in the quality of the finished product.

Detailed Method of Manufacturing Quinoa Protein Concentrate:

Pre-Conditioning:

The term “pre-conditioning” is used herein to indicate a step of treatment to remove saponins in the quinoa grain. Saponins are concentrated in the pericarp of quinoa. Saponin removal can be achieved via mechanical abrasion or washing, and/or a combination of both. Pre-conditioning of quinoa grain is described more fully above with respect to quinoa germ as in Example 3.

Conditioning:

The term “conditioning”, or “conditioned”, is used herein to indicate a step of treatment such as tempering or regulating the moisture content. The moisture content can be adjusted by the addition or removal of water. A preferred conditioning technique includes drying grain or evaporating water immediately after pre-conditioning in the range of about 12 to 30% moisture content of grain, preferably about 13 or about 14%. A preferred drying method includes evaporation with vacuum or air circulation through grain at a temperature range of 0-300° C., preferably ambient temperature. Conditioning of pre-conditioned quinoa grain is described more fully above with respect to quinoa germ as in Example 3.

Comminuting:

The term “comminuting”, or “comminuted”, is used herein to indicate a step of treatment such as milling with break or corrugated rolls and/or reduction or smooth rolls. A preferred comminuting technique includes passing the conditioned grain through a motorized mill with 2 corrugated rollers with a gap setting of 0.025 inch, followed by passing the grain through 2 corrugated rollers with a gap setting of 0.015 inch, followed by passing the grain through 2 corrugated rollers with a gap setting of 0.010 inch, followed by passing the grain through 2 smooth rollers with a gap setting of about 0.008 inch or less. Comminuting of conditioned quinoa grain is described more fully above with respect to quinoa germ as in Example 3.

Separating:

The term “separating”, or “separation” or “separate”, is used herein to indicate a step of treatment such as sieving, whereby the germ-rich fraction is separated from the peri-sperm rich fraction. A preferred separation technique includes passing the comminuted grain material through a motorized sifter with a screen made from stainless steel woven wire cloth 18×18 mesh, 0.015″ wire diameter, followed by sifting the material that passes through the 18×18 mesh screen through a screen made from stainless steel woven wire cloth with 30×30 mesh, 0.0095″ wire diameter, collecting the material that is retained on both screens as the quinoa germ-, bran-, or embryo-rich fraction of the invention, and collecting the material that passes through the 30×30 mesh screen as the perisperm-rich fraction, or quinoa flour. Separating the comminuted quinoa is described more fully above with respect to quinoa germ as in Example 3.

Protein Concentration:

Protein bodies are found in the quinoa germ in higher proportion in comparison to the perisperm-rich fraction. Quinoa protein or QPC of the invention can be readily obtained from quinoa germ by employing manipulations such as extraction, purification, iso-electric precipitation, ultra-filtration, or concentration, which are well known in the art. Additional protein methodology is discussed in Example 1, above. Alternatively, residual protein is found in the perisperm-rich fraction, or flour, and may be extracted from the flour (such as during the manufacturing of starch). It will be understood by those skilled in the art that the process disclosed herein can be operated with appropriate modifications and variations to obtain the afore-mentioned products. The process disclosed herein is designed to separate the germ-rich fraction from the perisperm-rich fraction contained in quinoa grain, in a manner that enables one to obtain edible quinoa protein concentrate from the germ-rich fraction as the starting material, as illustrated in FIG. 6, by which the method of the invention can be practiced.

### Example 6

### Methods for Manufacturing Edible Quinoa Fiber

In a sixth aspect the present invention provides methods of manufacturing edible fiber of quinoa. Also provided are the quinoa fiber produced according to the novel method, as well as numerous uses of the quinoa fiber produced according to this method.

Novel quinoa fiber is prepared from the germ-, bran-, or embryo-rich fraction of quinoa grain (“quinoa germ”) from quinoa (Chenopodium quinoa) grain using methods disclosed herein. There is no commercially-available, edible quinoa fiber today. The method described herein for the manufacture of quinoa fiber is designed with consideration of the physical structure of quinoa grain and for industry-scale production of edible fiber. The method described herein is designed to use the germ-, bran-, or embryo-rich fraction of quinoa grain as the starting material for the manufacture of quinoa fiber in order to maximize cost-efficient production versus producing fiber from whole quinoa grain as the starting material. The method is capable of producing a quinoa fiber preparation having at least 25 wt % fiber on a dry weight basis.

The quinoa fiber of the invention can be manufactured from non-genetically engineered quinoa grain and therefore is particularly useful in natural and/or organic labeled products. The quinoa fiber of the invention, while it may contain residual hypo-allergenic proteins, does not contain allergenic proteins such as those found in products derived from tree nuts, peanuts, and soybeans. It is therefore particularly useful in foods designed to replace edible fiber containing residual allergenic proteins (for example, wheat fiber or oat fiber which may be co-mingled with wheat). The quinoa fiber contains soluble and insoluble dietary fiber. Therefore, the quinoa fiber is useful as a food ingredient and dietary supplement to provide dietary fiber as well as necessary functionality in a variety of food products and pet food products.

The edible quinoa fiber produced according to the methods of the invention has numerous applications. For example, the quinoa fiber of the invention can be used in a wide variety of products including but not limited to bakery products, tortillas, crackers, granolas, toppings, batters and coatings, confections, breakfast cereal, instant and cook-up porridge, pudding or tapioca-like pudding, yogurt, ice-cream, cheese, cream preparations, coffee whiteners and non-dairy creamers, whipped toppings, snack and extruded foods, frozen foods, beverages, milk-like beverages, powdered drink beverages, ready-to-eat packaged foods, fruit preparations, salad dressing, frozen foods, foods for infants and toddlers, infant formula, nutrition bars, meat extenders, meat and seafood analogs, emulsified meats, canned meats, whole muscle and coarsely chopped meat products, seafood and poultry products, pastas, dough, candy and confections, sauces, seasoning and dry ingredient mixes. Additional uses in food include but not limited to viscosity control, binder, stabilizer, moisture control, to reduce calories, to improve mouth-feel and texture, fried foods, encapsulation, in production of low-glycemic and low-carb foods, anti-caking agent. Additional non-food uses include but not limited to personal care products, soaps, candles, hair spray, conditioners, shampoo, mousses, lotions, sunscreens and skin care preparations, cosmetics and nutraceuticals, textiles, paper, packaging, construction, insulation, encapsulation, cleansers, biofuels, paints, and anti-foaming agents.

The quinoa fiber of the invention is particularly useful as a nutritional and dietary supplement to provide insoluble and soluble dietary fiber in a food or beverage product, or alone. The quinoa fiber of the invention is also particularly useful as a replacement for genetically engineered and allergenic sources of vegetable fiber.

The quinoa fiber can be used in milk-free and dairy-free, soy-free, nut-free, peanut-free, gluten-free, and egg-free food or cosmetic products intended for use in subjects who require less- or hypo-allergenic food products. The quinoa fiber can be used in plant-based, vegan, low-glycemic, low-carb, vegetarian, non GMO and nongenetically engineered food or cosmetic products. In addition, quinoa fiber can serve as a nutritious ingredient in pet foods and animal feeds, such as cattle feed, since the FDA banned the use of animal protein in cattle feed as a preventative measure against bovine spongiform encephalopathy (i.e. BSE or mad cow disease) [DEPARTMENT OF HEALTH AND HUMAN SERVICES (2004), Food and Drug Administration, 21 CFR Parts 189 and 700, [Docket No. 2004N-0081], RIN-0910-AF47, Use of Materials Derived From Cattle in Human Food and Cosmetics].

Overview of Method of Manufacturing Edible Quinoa Fiber:

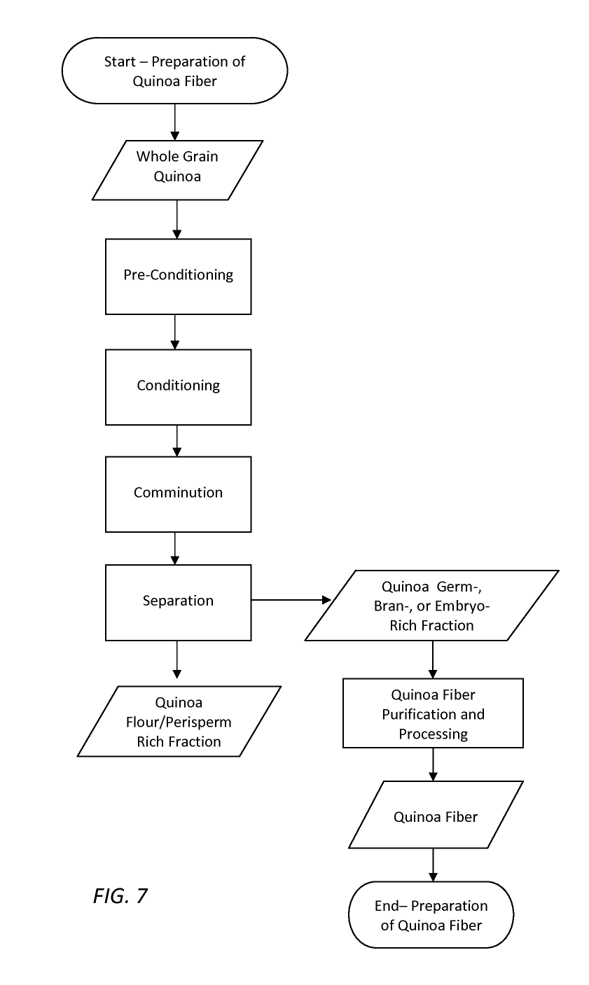

The present invention provides a non-obvious method for manufacturing edible quinoa fiber. The method can be characterized by the steps of: 1) pre-conditioning the quinoa grain; 2) conditioning the quinoa grain; 3) comminuting the quinoa grain; 4) separating the fraction containing a majority of the germ from the fraction containing a majority of the perisperm; and 5) fiber production from the quinoa germ. Thus, a quinoa germ is obtained as in Example 3, above, that can be used as the starting material for the fiber production, which can be accomplished via purification, sieving, filtering, flocculation, centrifuging, mechanical pressing, supercritical gas-, or liquid-extraction, air-classification, aspiration. FIG. 7 illustrates the steps outlined above. The quinoa fiber can be further treated by refining, deodorizing, bleaching, enzyme modification, and/or chemical hydrolysis if necessary. Quinoa grain can be sorted by size, shape, or color prior to pre-conditioning, conditioning, or comminution to aid in the quality of the finished product.

Detailed Method of Manufacturing Edible Quinoa Fiber:

Pre-Conditioning:

The term “pre-conditioning” is used herein to indicate a step of treatment to remove saponins in the quinoa grain. Saponins are concentrated in the pericarp of quinoa. Saponin removal can be achieved via mechanical abrasion or washing, and/or a combination of both. Pre-conditioning of quinoa grain is described more fully above with respect to quinoa germ as in Example 3.

Conditioning:

The term “conditioning”, or “conditioned”, is used herein to indicate a step of treatment such as tempering or regulating the moisture content. The moisture content can be adjusted by the addition or removal of water. A preferred conditioning technique includes drying grain or evaporating water immediately after pre-conditioning in the range of about 12 to 30% moisture content of grain, preferably about 13 or about 14%. A preferred drying method includes evaporation with vacuum or air circulation through grain at a temperature range of 0-300° C., preferably ambient temperature. Conditioning of pre-conditioned quinoa grain is described more fully above with respect to quinoa germ as in Example 3.

Comminuting:

The term “comminuting”, or “comminuted”, is used herein to indicate a step of treatment such as milling with break or corrugated rolls and/or reduction or smooth rolls. A preferred comminuting technique includes passing the conditioned grain through a motorized mill with 2 corrugated rollers with a gap setting of 0.025 inch, followed by passing the grain through 2 corrugated rollers with a gap setting of 0.015 inch, followed by passing the grain through 2 corrugated rollers with a gap setting of 0.010 inch, followed by passing the grain through 2 smooth rollers with a gap setting of about 0.008 inch or less. Comminuting of conditioned quinoa grain is described more fully above with respect to quinoa germ as in Example 3.

Separating:

The term “separating”, or “separation” or “separate”, is used herein to indicate a step of treatment such as sieving, where the germ-rich fraction is separated from the peri-sperm rich fraction. The preferred separation technique includes passing the comminuted grain material through a motorized sifter with a screen made from stainless steel woven wire cloth 18×18 mesh, 0.015″ wire diameter, followed by sifting the material that passes through the 18×18 mesh screen through a screen made from stainless steel woven wire cloth with 30×30 mesh, 0.0095″ wire diameter, collecting the material that is retained on both screens as the quinoa germ-, bran-, or embryo-rich fraction of the invention, and collecting the material that passes through the 30×30 mesh screen as the perisperm-rich fraction, or quinoa flour. Separating the comminuted quinoa is described more fully above with respect to quinoa germ as in Example 3.

Fiber Production:

Quinoa fiber of the invention can be readily obtained from quinoa germ by employing manipulations such as purification, mechanical pressing, supercritical gas-, or solvent-extraction which are well known in the art. It will be understood by those skilled in the art that the process disclosed herein can be operated with appropriate modifications and variations to obtain the afore-mentioned products. The process disclosed herein is designed to separate the germ-rich fraction from the perisperm-rich fraction contained in quinoa grain, in a manner that enables one to obtain edible quinoa fiber from the germ-rich fraction as the starting material as discussed above and illustrated in FIG. 7.

### Example 7

### Methods for Manufacturing Unmodified Quinoa Starch

In a seventh aspect the present invention provides methods of manufacturing starch of quinoa. Also provided are the quinoa starch produced according to the novel method, as well as numerous uses of the quinoa starch produced according to this method.

As addressed above, aspects of the invention provide native (unmodified) starch of quinoa and uses of this starch. The quinoa starch is prepared from quinoa (Chenopodium quinoa) grain. The method described herein for the manufacture of quinoa starch of the invention is designed with consideration of the physical structure of quinoa grain and for industry-scale production of edible starch. The method employs the perisperm-rich fraction as the starting material for the manufacture of quinoa starch in order to maximize cost-efficient production versus producing starch from whole quinoa grain as the starting material.

The quinoa starch of the invention is manufactured from non-genetically engineered quinoa grain and therefore is particularly useful in natural and/or organic labeled products. The quinoa starch of the invention contains hypo-allergenic proteins, as opposed to allergenic proteins from wheat, barley, and corn. Therefore, quinoa starch is useful as a hypo-allergenic and gluten free ingredient to provide necessary functionality or nonfunctionality (such as a bulking agent) in a variety of food products, pet food products, cosmetics, and pharmaceuticals.

The edible quinoa starch produced according to the methods of the invention has numerous applications. For example, the quinoa starch of the invention can be used in a wide variety of products including but not limited to bakery products, tortillas, crackers, granolas, toppings, batters and coatings, confections, breakfast cereal, instant and cook-up porridge, pudding or tapioca-like pudding, yogurt, ice-cream, cheese, cream preparations, snack and extruded foods, frozen foods, beverages, milk-like beverages, ready-to-eat packaged foods, fruit preparations, salad dressing, foods for infants and toddlers, nutrition bars, meat extenders, meat analogs, pastas, dough, candy and confections, sauces, seasoning and dry ingredient mixes. Additional uses in food include but are not limited to binders, stabilizers, thickeners, viscosity control, anti-caking agent, to improve mouth-feel and texture, coating agents for fried and baked foods, to reduce the caloric content or in the production of low-glycemic foods. Additional non-food uses include ethanol, paper, encapsulation, adhesives, films, releasing agents, packaging, personal care products, hair spray, conditioners, shampoo, mousses, lotions, sunscreens and skin care preparations, and cosmetics.

The quinoa starch of the invention is particularly useful as an encapsulating agent, carrier and/or tablet agent in the preparation of food flavors, cosmetic fragrances and pharmaceuticals. The quinoa starch of the invention is particularly useful as a dusting agent for surgical gloves. The quinoa starch of the invention is particularly useful as a replacement for genetically engineered cornstarch and as an ingredient or starting material for the manufacture of starch-based ingredients. The quinoa starch of the invention can also be used as a source of nongenetically engineered starting material for the manufacture of dextrose, syrup, high fructose syrup, maltodextrin, dextrins, and ethanol.

The quinoa starch can be used in milk-free and dairy-free, soy-free, nut-free, peanut-free, gluten-free, and egg-free food or cosmetic products intended for use in subjects who require less- or hypo-allergenic food products. The quinoa starch can be used in plant-based, vegan, low-glycemic, vegetarian, non-GMO and non-genetically engineered food or cosmetic products. In addition, quinoa starch containing residual and hypo-allergenic protein can serve as an ingredient in pet foods and animal feeds, such as cattle feed, since the FDA banned the use of animal protein in cattle feed as a preventative measure against bovine spongiform encephalopathy (i.e., BSE or mad cow disease) [DEPARTMENT OF HEALTH AND HUMAN SERVICES (2004), Food and Drug Administration, 21 CFR Parts 189 and 700, [Docket No. 2004N-0081], RIN-091 0-AF47, Use of Materials Derived From Cattle in Human Food and Cosmetics].

Overview of Method of Manufacturing Quinoa Starch:

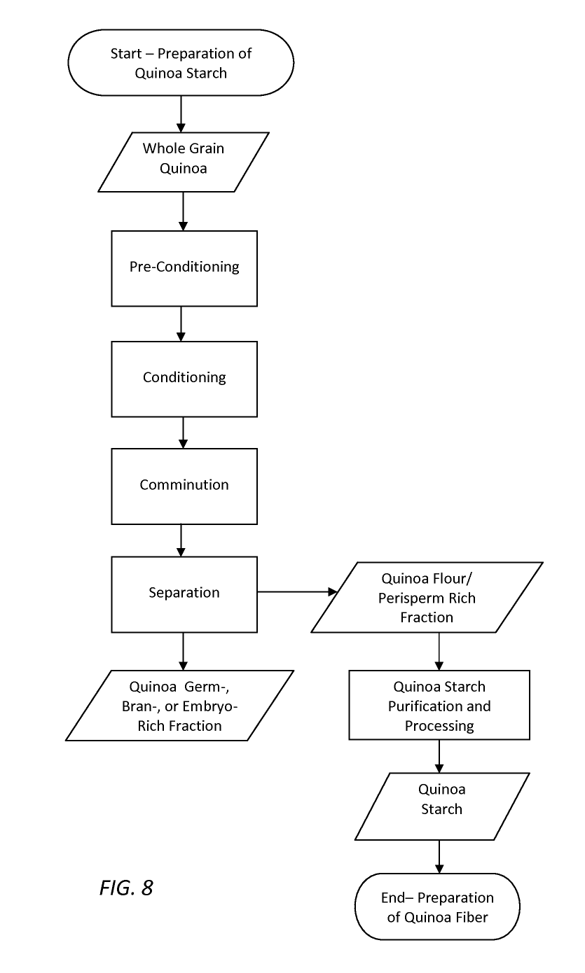

The present invention provides a non-obvious method for manufacturing edible quinoa fiber. The method can be characterized by the steps of: 1) pre-conditioning the quinoa grain; 2) conditioning the quinoa grain; 3) comminuting the quinoa grain; 4) separating the fraction containing a majority of the germ from the fraction containing a majority of the perisperm; and 5) starch purification/extraction from the perisperm fraction. FIG. 8 illustrates the steps outlined above. Thus, a quinoa flour is obtained as in Example 2, above, that can be used as the starting material for the starch production, which can be accomplished via purification or extraction. The quinoa starch can be further treated by particle size reduction, drying, bleaching, traditional chemical and physical modification of starch, agglomeration, pre-gelatinization, enzyme modification, and/or hydrolysis if necessary. Quinoa grain can be sorted by size, shape, or color prior to pre-conditioning, conditioning, or comminution to aid in the quality of the finished product.

Detailed Method of Manufacturing Quinoa Starch:

Pre-Conditioning:

The term “pre-conditioning” is used herein to indicate a step of treatment to remove saponins in the quinoa grain. Saponins are concentrated in the pericarp of quinoa. Saponin removal can be achieved via mechanical abrasion or washing, and/or a combination of both. Pre-conditioning of quinoa grain is described more fully above with respect to quinoa flour as in Example 2, above.

Conditioning:

The term “conditioning”, or “conditioned”, is used herein to indicate a step of treatment such as tempering or regulating the moisture content. The moisture content can be adjusted by the addition or removal of water. A preferred conditioning technique includes drying grain or evaporating water immediately after pre-conditioning in the range of about 12 to 30% moisture content of grain, preferably about 13 or about 14%. A preferred drying method includes evaporation with vacuum or air circulation through grain at a temperature range of 0-300° C., preferably ambient temperature. Conditioning of pre-conditioned quinoa grain is described more fully above with respect to quinoa flour as in Example 2.

Comminuting:

The term “comminuting”, or “comminuted”, is used herein to indicate a step of treatment such as milling with break or corrugated rolls and/or reduction or smooth rolls. A preferred comminuting technique includes passing the conditioned grain through a motorized mill with 2 corrugated rollers with a gap setting of 0.025 inch, followed by passing the grain through 2 corrugated rollers with a gap setting of 0.015 inch, followed by passing the grain through 2 corrugated rollers with a gap setting of 0.010 inch, followed by passing the grain through 2 smooth rollers with a gap setting of about 0.008 inch or less. Comminuting of conditioned quinoa grain is described more fully above with respect to quinoa flour as in Example 2.

Separating:

The term “separating”, or “separation” or “separate”, is used herein to indicate a step of treatment such as sieving, whereby the germ-rich fraction is separated from the peri-sperm rich fraction. The preferred separation technique includes passing the comminuted grain material through a motorized sifter with a screen made from stainless steel woven wire cloth 18×18 mesh, 0.015″ wire diameter, followed by sifting the material that passes through the 18×18 mesh screen through a screen made from stainless steel woven wire cloth with 30×30 mesh, 0.0095″ wire diameter, collecting the material that is retained on both screens as the germ-, bran-, or embryo-rich fraction, and collecting the material that passes through the 30×30 mesh screen as the perisperm-rich fraction, or quinoa flour. Steps 1, 2, 3, and 4 (i.e. pre-conditioning, conditioning, comminuting and separating) practiced in this manner will produce a germ-rich fraction and a peri-sperm rich fraction, or quinoa flour. Alternatively, separation can occur prior to smooth rolling because embryo-rich fraction is lighter and the peri-sperm rich fraction is denser. Alternatively, the peri-sperm rich fraction does not have to go through smooth rollers and can remain coarse, such as that of semolina flour. The flour has reduced enzyme activity and increased shelf life because it is separated from embryo-rich fraction containing increased levels of metabolically active enzymes that can lead to product deterioration and off flavors. Alternatively, the number of screens used in the sifter, the mesh size of the screens, the wire diameter, the micron openings, and the material of the screens can be adjusted. Alternatively, separation can include but not limited to aspiration, air classification, pneumatic pressure, vacuum, and/or vibration.

Starch Extraction:

Quinoa starch of the invention can be readily obtained from quinoa flour by employing manipulations such as separation, extraction, purification, or concentration. It will be understood by those skilled in the art that the process disclosed herein can be operated with appropriate modifications and variations to obtain the afore-mentioned products. The process disclosed herein is designed to separate the germ-rich fraction from the perisperm-rich fraction contained in quinoa grain, in a manner that enables one to obtain edible quinoa starch from the perisperm-rich fraction as the starting material as discussed above and illustrated in FIG. 8.

All references cited in the present application are incorporated in their entirety herein by reference to the extent not inconsistent herewith.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of processing quinoa grain comprising the steps of:
pre-conditioning the quinoa grain;
conditioning the pre-conditioned quinoa grain;
comminuting the conditioned quinoa grain; and
separating the comminuted quinoa grain, whereby the separation step yields a germ-rich fraction and a peri-sperm rich fraction.

2. The method according to claim 1 wherein the pre-conditioning is selected from the group consisting of mechanical abrasion, washing the quinoa grain and combinations thereof.

3. The method according to claim 1 wherein pre-conditioning includes abrasion followed by an initial quick wash with stiffing, agitation, spray or counter current extraction followed by draining or centrifugation, whereby the quick wash minimizes penetration of the water-soluble saponins into the grain.

4. The method according to claim 1 wherein the pre-conditioning includes abrasion followed by a plurality of quick washes.

5. The method according to claim 4 wherein one or more of the plurality of quick washes include stirring, agitation, spray or counter current extraction followed by draining or centrifugation.

6. The method according to claim 4 wherein the first of the plurality of quick washes employs a residence time of about 30 seconds to about 2 minutes and a subsequent wash employs a residence time of about 2 minutes to about 10 minutes.

7. The method according to claim 1 wherein the pre-conditioning is selected from the group consisting of mechanical abrasion, washing, polishing, peeling, aspiration, air classification, sieving, pneumatic pressure, vacuum, nixtamalization, rinsing, solvent leaching the quinoa grain and combinations thereof.

8. The method according to claim 1 wherein the conditioning is performed by evaporating water from the grain to yield grain with a moisture content of about 12 to about 30%.

9. The method according to claim 8 wherein the grain is conditioned to a moisture content of about 13 to about 14%.

10. The method according to claim 1 further comprising the step of isolating the quinoa protein from the germ-rich fraction.

11. The method according to claim 10 wherein the quinoa protein is isolated from the germ-rich fraction using a technique selected from the group consisting of extraction, purification, iso-electric precipitation, ultra-filtration and concentration.

12. The method according to claim 11 further comprising the step of clarifying the extracted protein to remove quinoa fiber or impurities.

13. The method according to claim 12 wherein the extracted protein is clarified by centrifugation.

14. The method according to claim 11 further comprising the step of neutralizing the extracted protein.

15. The method according to claim 14 further comprising the steps of: precipitating the neutralized protein;
isolating the precipitated protein; and
neutralizing the precipitated protein.

16. The method according to claim 1 wherein the conditioned quinoa grain is comminuted using a technique selected from the group consisting of polishing, abrasion, milling, pin milling, hammer milling, degerming, stone grinding, cracking, crushing, slicing, flaking and combinations thereof.

17. The method according to claim 1 wherein the comminuted quinoa grain is separated using a technique selected from the group consisting of sieving, aspiration, air classification, pneumatic pressure, vacuum, vibration and combinations thereof.

18. The method according to claim 1 further comprising the step of isolating the quinoa oil from the germ-rich fraction.

19. The method according to claim 18 wherein the quinoa oil is isolated from the germ-rich fraction using a technique selected from the group consisting of purification, mechanical pressing, supercritical gas-extraction, solvent-extraction and combinations thereof.

20. The method according to claim 18 further comprising the step of treating the isolated quinoa oil by refining, deodorizing, bleaching, enzyme modification, chemical hydrolysis and combinations thereof.

21. The method according to claim 1 further comprising the step of isolating the quinoa fiber from the germ-rich fraction.

22. The method according to claim 21 wherein the quinoa fiber is isolated from the germ-rich fraction using a technique selected from the group consisting of purification, sieving, filtering, flocculation, centrifuging, mechanical pressing, supercritical gas-extraction, or liquid-extraction, air-classification, aspiration and combinations thereof.

23. The method according to claim 21 further comprising the step of treating the isolated quinoa fiber by refining, deodorizing, bleaching, enzyme modification, chemical hydrolysis and combinations thereof.

24. The method according to claim 1 further comprising the step of isolating the quinoa starch from the perisperm-rich fraction.

25. The method according to claim 24 wherein the quinoa starch is isolated from the perisperm-rich fraction using a technique selected from the group consisting of purification, extraction, and combinations thereof.

26. The method according to claim 24 further comprising the step of treating the isolated quinoa starch by refining, deodorizing, bleaching, enzyme modification, chemical hydrolysis and combinations thereof.

27. A method of processing quinoa grain comprising the steps of:
comminuting the quinoa grain; and
extracting the protein from the comminuted quinoa grain using an alkaline solution, whereby the protein becomes solubilized from the comminuted quinoa grain.

28. The method according to claim 27 further comprising the step of clarifying the extracted protein to remove quinoa fiber or impurities.

29. The method according to claim 28 wherein the extracted protein is clarified by centrifugation.

30. The method according to claim 27 further comprising the step of neutralizing the extracted protein.

31. The method according to claim 30 further comprising the steps of:
precipitating the neutralized protein;
isolating the precipitated protein; and
neutralizing the precipitated protein.

32. The method according to claim 27 further comprising the step of extracting oil from the quinoa grain.

33. The method according to claim 32 wherein the oil is extracted following the comminuting step.

34. The method according to claim 27 further comprising the steps of:
pre-conditioning the quinoa grain; and
conditioning the pre-conditioned quinoa grain prior to the comminuting step.

35. The method according to claim 27 further comprising the step of separating the comminuted quinoa grain prior to extraction, whereby the separation step yields a germ-rich fraction and a perisperm-rich fraction.

36. The method according to claim 35 further comprising the step of isolating the quinoa protein from the germ-rich fraction.

37. The method according to claim 36 wherein the quinoa protein is isolated from the germ-rich fraction using a technique selected from the group consisting of extraction, purification, iso-electric precipitation, ultra-filtration and concentration.

38. The method according to claim 34 wherein the pre-conditioning is selected from the group consisting of mechanical abrasion, washing the quinoa grain and combinations thereof.

39. The method according to claim 38 wherein the pre-conditioning includes abrasion followed by a plurality of quick washes.

40. The method according to claim 34 wherein the conditioning is performed by evaporating water from the grain to yield grain with a moisture content of about 12 to about 30%.

41. The method according to claim 40 wherein the grain is conditioned to a moisture content of about 13 to about 14%.

42. The method according to claim 35 further comprising the step of isolating the quinoa oil from the germ-rich fraction.

43. The method according to claim 35 further comprising the step of isolating the quinoa fiber from the germ-rich fraction.

44. The method according to claim 35 further comprising the step of isolating the quinoa starch from the perisperm-rich fraction.

45. The method according to claim 27 further comprising the step of isolating the quinoa oil from the extracted protein.

46. The method according to claim 27 further comprising the step of isolating the quinoa fiber from the extracted protein.

47. The method according to claim 27 further comprising the step of isolating the quinoa starch from the by-product of the extracted protein.

48. A method of processing quinoa grain comprising the steps of:
pre-conditioning the quinoa grain;
conditioning the pre-conditioned quinoa grain;
comminuting the conditioned quinoa grain;
separating the comminuted quinoa grain, whereby the separation step yields a germ-rich fraction and a perisperm-rich fraction; and
isolating the quinoa protein from the germ-rich fraction.

49. The method according to claim 48 wherein the quinoa protein is isolated from the germ-rich fraction using a technique selected from the group consisting of extraction, purification, iso-electric precipitation, ultra-filtration and concentration.

50. The method according to claim 49 further comprising the step of clarifying the extracted protein to remove quinoa fiber or impurities.

51. The method according to claim 50 wherein the extracted protein is clarified by centrifugation.

52. The method according to claim 49 further comprising the step of neutralizing the extracted protein.

53. The method according to claim 52 further comprising the steps of: precipitating the neutralized protein;
isolating the precipitated protein; and
neutralizing the precipitated protein.

54. The method according to claim 48 further comprising the step of collecting the perisperm-rich fraction, whereby the collected perisperm-rich fraction can be further processed for use as quinoa flour or quinoa starch.
